(19) United States
(12) Patent Application Publication
(10) Pub. No.: US 2026/0301970 A1
(43) Pub. Date: Oct. 1, 2026

(54) SYSTEM, METHOD, AND COMPUTER PROGRAM PRODUCT FOR PLATFORM FOR DEVELOPMENT AND DEPLOYMENT OF ARTIFICIAL INTELLIGENCE BASED SOFTWARE AS MEDICAL DEVICE

(71) Applicant: Bayer HealthCare LLC, Whippany, NJ (US)

(72) Inventors: Markus BLANK, Glienicke/Nordbalm (DE); Steffen VOGLER, Berlin (DE); Tobias ROST, Berlin (DE); Guido MATTHEWS, Schöneiche (DE)

(21) Appl. No.: 19/481,816

(22) PCT Filed: May 16, 2024
(86) PCT No.: PCT/US2024/029620

§ 371 Date: Nov. 5, 2025

Related U.S. Application Data
(60) Provisional application No. 63/503,347, filed on May 19, 2023.

(51) Int. Cl. G16H 50/70; G06F 16/25; G16H 10/60; G16H 50/20

(52) U.S. Cl. CPC G16H 50/70; G06F 16/254; G16H 10/60; G16H 50/20

(57) ABSTRACT

Provided is a system for generation of artificial intelligence (AI) based automated healthcare applications that includes an AI medical device platform that comprises at least one processor in communication with a data repository system, the at least one processor configured to receive formatted data associated with a plurality of medical procedures for generation of one or more machine 2024/242985 learning models, receive data associated with one or more regulatory guidance and approval processes, generate one or more machine learning models configured to predict one or more aspects associated with a medical procedure based on the data associated with the plurality of medical procedures and the data associated with the regulatory guidance and approval process, and deploy the one or more machine learning models to a production environment based on generating the one or more machine learning models. Methods and computer program products are also disclosed.

### CROSS-REFERENCE TO RELATED APPLICATIONS

This application claims the benefit of U.S. Provisional Application No. 63/503,347, filed May 19, 2023, the entire disclosure of which is hereby incorporated by reference in its entirety.

### BACKGROUND

### 1. Field

This disclosure relates generally to managing health informatics applications and, in some non-limiting embodiments, to systems, methods, and computer program products that provide a platform for development and deployment of automated healthcare applications, including artificial intelligence (AI) based software as medical devices.

### 2. Technical Considerations

Artificial intelligence (AI) may be used in healthcare as a way to mimic human cognition in analysis, presentation, and/or comprehension of healthcare data. AI may describe the ability of computer programs, such as computer algorithms, to approximate conclusions based on input data, which may be medical data. In some instances, computer algorithms can be used to recognize patterns in data and create logic for identifying such patterns. Such computer algorithms may include machine learning models that are trained to perform certain tasks using extensive amounts of input data. In some instances, healthcare based computer algorithms may include machine learning models configured for providing outputs with regard to medical procedures.

Software as a medical device (SaMD) may refer to software intended to be used for one or more medical purposes that performs these purposes without necessarily being installed as part of a hardware medical device. In some instances, SaMDs may include machine learning models specifically designed to provide outputs with regard to a medical procedure for which the SaMD is designed to provide a result, such as a prediction of diagnosis. For example, SaMDs may range from software that allows a smartphone to view images obtained from a magnetic resonance imaging (MRI) medical device for diagnostic purposes to Computer-Aided Detection (CAD) software that performs image post-processing to help detect breast cancer.

However, building SaMDs, and the machine learning models that are included therewith, may require large amounts of data. Accordingly, manually programmed processes that are developed for providing the input data to the machine learning models may require intensive amounts of network resources, may require constant manual updating, and may be inaccurate. In addition, datasets that are used for generation of machine learning models may be stored in various different locations and may require significant effort to retrieve and use for generation of models. Furthermore, SaMDs may require regulatory guidance and approval at the various stages of development and deployment. Determining whether SaMDs meet the applicable regulatory guidance and approval may require extensive resources for retrieving and storing applicable regulations prior to and during development, as well extensive resources for tracking when SaMDs are deployed.

### SUMMARY

Accordingly, disclosed are systems, methods, and computer program products that provide for a platform for development and deployment of automated healthcare applications.

Clause 1: A system for generation of artificial intelligence (AI) based automated healthcare applications, comprising: a data repository system, wherein the data repository system stores at least one of the following: formatted data associated with a plurality of medical procedures for generation of one or more machine learning models, data associated with a regulatory guidance and approval process, or any combination thereof; an AI medical device platform that comprises at least one processor in communication with the data repository system, the at least one processor configured to: receive the formatted data associated with a plurality of medical procedures for generation of one or more machine learning models; receive the data associated with one or more regulatory guidance and approval processes; generate one or more machine learning models configured to predict one or more aspects associated with a medical procedure based on the formatted data associated with the plurality of medical procedures and the data associated with the regulatory guidance and approval process; deploy the one or more machine learning models to a production environment based on generating the one or more machine learning models.

Clause 2: The system of clause 1, wherein the data repository system comprises: a data ingestion subsystem to receive data from a plurality of data sources; a data storage subsystem to store data for the data repository system; a data processing subsystem to transform the data of the plurality of data sources based on extract, transform, load (etl) pipelines to provide transformed data to be used for generation of the one or more machine learning models; a data analytics subsystem to provide access to data visualization tools and database analytics tools to be used for generation of the one or more machine learning models; and a data cloud foundation subsystem to provide one or more rules associated with control operations for the data repository system.

Clause 3: The system of clauses 1 or 2, wherein the data repository system is configured to: receive initial data associated with one or more data records from a plurality of data sources, wherein the plurality of data sources comprises at least one of the following: an electronic medical record (EMR) system; a medical imaging system; a fluid injection system; a pathology information system; a laboratory information system; or any combination thereof; and perform one or more data curation procedures on the initial data associated with one or more data records to provide the formatted data associated with a plurality of medical procedures for generation of one or more machine learning models.

Clause 4: The system of any of clauses 1-3, wherein, when generating one or more machine learning models configured to predict one or more aspects associated with a medical procedure, the at least one processor is configured to: train the one or more machine learning models based on the formatted data associated with a plurality of medical procedures for generation of one or more machine learning models to provide one or more trained machine learning models; and validate an output of the one or more trained machine learning models based on the data associated with the regulatory guidance and approval process.

Clause 5: The system of any of clauses 1-4, wherein the at least one processor is further configured to: receive a request for access to a particular machine learning model of a plurality of machine learning models; determine whether the request for access complies with one or more criteria associated with access to the particular machine learning model; and provide access to the particular machine learning model based on determining that the request for access complies with one or more criteria associated with access to the particular machine learning model.

Clause 6: The system of any of clauses 1-5, wherein the formatted data associated with a plurality of medical procedures for generation of one or more machine learning models comprises a plurality of datasets, wherein each dataset of the plurality of datasets is associated with a particular medical procedure of the plurality of medical procedures, wherein the at least one processor is further configured to: receive a request for access to a particular dataset of a plurality of datasets; determine whether the request for access complies with one or more criteria associated with access to the particular dataset; and provide access to the particular dataset based on determining that the request for access complies with one or more criteria associated with access to the particular dataset.

Clause 7: The system of any of clauses 1-6, wherein, when providing access to the particular dataset, the at least one processor is configured to: perform an application programming interface (API) call associated with the particular dataset to the data repository system to provide access to the particular dataset.

Clause 8: The system of any of clauses 1-7, wherein the formatted data associated with a plurality of medical procedures for generation of one or more machine learning models comprises a plurality of datasets, wherein each dataset of the plurality of datasets is associated with a particular medical procedure of the plurality of medical procedures, wherein the at least one processor is further configured to: train the one or more machine learning models based on a first dataset of the plurality of datasets to provide one or more trained machine learning models; assign a unique identifier to the first dataset based on training the one or more machine learning models; assign the unique identifier to the one or more trained machine learning models; and validate an output of the one or more trained machine learning models based on the data associated with the regulatory guidance and approval process, wherein when validating the output of the one or more trained machine learning models, the at least one processor is configured to: validate the first dataset and the one or more trained machine learning models based on the unique identifier assigned to the first dataset and the one or more trained machine learning models.

Clause 9: The system of any of clauses 1-8, wherein, when deploying the one or more machine learning models to the production environment, the at least one processor is configured to: deploy the one or more machine learning models to at least one of the following: a production environment of a third-party platform that is not associated with the AI medical device platform; a production environment of the AI medical device platform; a platform that includes a standardized container; or any combination thereof.

Clause 10: A method for generation of artificial intelligence (AI) based automated healthcare applications, comprising: receiving, with at least one processor of an AI medical device platform, formatted data associated with a plurality of medical procedures for generation of one or more machine learning models from a data repository system; receiving, with the at least one processor, data associated with one or more regulatory guidance and approval processes; generating, with the at least one processor, one or more machine learning models configured to predict one or more aspects associated with a medical procedure based on the formatted data associated with a plurality of medical procedures for generation of one or more machine learning models and the data associated with the regulatory guidance and approval process; and deploying, with the at least one processor, the one or more machine learning models to a production environment based on generating the one or more machine learning models.

Clause 11: The method of clause 10, further comprising: receiving initial data associated with one or more data records from a plurality of data sources, wherein the plurality of data sources comprises at least one of the following: an electronic medical record (EMR) system; a medical imaging system; a fluid injection system; a pathology information system; a laboratory information system; any combination thereof.

Clause 12: The method of clauses 10 or 11, further comprising: performing one or more data curation procedures on the initial data associated with one or more data records to provide the formatted data associated with a plurality of medical procedures for generation of one or more machine learning models.

Clause 13: The method of any of clauses 10-12, wherein generating one or more machine learning models configured to predict one or more aspects associated with a medical procedure comprises: training the one or more machine learning models based on the formatted data associated with the plurality of medical procedures to provide one or more trained machine learning models; and validating an output of the one or more trained machine learning models based on the data associated with the regulatory guidance and approval process.

Clause 14: The method of any of clauses 10-13, further comprising: receiving a request for access to a particular machine learning model of a plurality of machine learning models; determining whether the request for access complies with one or more criteria associated with access to the particular machine learning model; and providing access to the particular machine learning model based on determining that the request for access complies with one or more criteria associated with access to the particular machine learning model.

Clause 15: The method of any of clauses 10-14, wherein the formatted data associated with a plurality of medical procedures for generation of one or more machine learning models comprises a plurality of datasets, wherein each dataset of the plurality of datasets is associated with a particular medical procedure of the plurality of medical procedures, and wherein the method further comprises: receiving a request for access to a particular dataset of a plurality of datasets; determining whether the request for access complies with one or more criteria associated with access to the particular dataset; and providing access to the particular dataset based on determining that the request for access complies with one or more criteria associated with access to the particular dataset.

Clause 16: The method of any of clauses 10-15, wherein providing access to the particular dataset comprises: performing an application programming interface (API) call associated with the particular dataset to the data repository system to provide access to the particular dataset.

Clause 17: The method of any of clauses 10-16, wherein the formatted data associated with a plurality of medical procedures for generation of one or more machine learning models comprises a plurality of datasets, wherein each dataset of the plurality of datasets is associated with a particular medical procedure of the plurality of medical procedures, and wherein the method further comprises: training the one or more machine learning models based on a first dataset of the plurality of datasets to provide one or more trained machine learning models; assigning a unique identifier to the first dataset based on training the one or more machine learning models; assigning the unique identifier to the one or more trained machine learning models; validating an output of the one or more trained machine learning models based on the data associated with the regulatory guidance and approval process, wherein validating the output of the one or more trained machine learning models comprises: validating the first dataset and the one or more trained machine learning models based on the unique identifier assigned to the first dataset and the one or more trained machine learning models.

Clause 18: The method of any of clauses 10-17, wherein deploying the one or more machine learning models to the production environment comprises: deploying the one or more machine learning models to at least one of the following: a production environment of a third-party platform that is not associated with the AI medical device platform; a production environment of the AI medical device platform; a platform that includes a standardized container; or any combination thereof.

Clause 19: A computer program product for generation of artificial intelligence (AI) based automated healthcare applications comprising at least one non-transitory computer-readable medium including program instructions that, when executed by at least one processor, cause the at least one processor to: receive formatted data associated with a plurality of medical procedures for generation of one or more machine learning models from a data repository system; receive data associated with one or more regulatory guidance and approval processes from the data repository system; generate one or more machine learning models configured to predict one or more aspects associated with a medical procedure based on the data associated with the plurality of medical procedures and the data associated with the regulatory guidance and approval process; and deploy the one or more machine learning models to a production environment based on generating the one or more machine learning models.

Clause 20: The computer program product of clause 19, wherein the program instructions further cause the at least one processor to: receive initial data associated with one or more data records from a plurality of data sources, wherein the plurality of data sources comprises at least one of the following: an electronic medical record (EMR) system; a medical imaging system; a fluid injection system; a pathology information system; a laboratory information system; any combination thereof.

Clause 21: The computer program product of clauses 19 or 20, wherein the program instructions further cause the at least one processor to: perform one or more data curation procedures on the initial data associated with one or more data records to provide the formatted data associated with a plurality of medical procedures for generation of one or more machine learning models.

Clause 22: The computer program product of any of clauses 19-21, wherein the program instructions that cause the at least one processor to generate one or more machine learning models configured to predict one or more aspects associated with a medical procedure cause the at least one processor to: train the one or more machine learning models based on the formatted data associated with a plurality of medical procedures for generation of one or more machine learning models to provide one or more trained machine learning models; and validate an output of the one or more trained machine learning models based on the data associated with the regulatory guidance and approval process.

Clause 23: The computer program product of any of clauses 19-22, wherein the program instructions further cause the at least one processor to: receive a request for access to a particular machine learning model of a plurality of machine learning models; determine whether the request for access complies with one or more criteria associated with access to the particular machine learning model; and provide access to the particular machine learning model based on determining that the request for access complies with one or more criteria associated with access to the particular machine learning model.

Clause 24: The computer program product of any of clauses 19-23, wherein the formatted data associated with a plurality of medical procedures for generation of one or more machine learning models comprises a plurality of datasets, wherein each dataset of the plurality of datasets is associated with a particular medical procedure of the plurality of medical procedures, wherein the program instructions further cause the at least one processor to: receive a request for access to a particular dataset of a plurality of datasets; determine whether the request for access complies with one or more criteria associated with access to the particular dataset; and provide access to the particular dataset based on determining that the request for access complies with one or more criteria associated with access to the particular dataset.

Clause 25: The computer program product of any of clauses 19-24, wherein the program instructions that cause the at least one processor to provide access to the particular dataset cause the at least one processor to: perform an application programming interface (API) call associated with the particular dataset to the data repository system to provide access to the particular dataset.

Clause 26: The computer program product of any of clauses 19-25, wherein the formatted data associated with a plurality of medical procedures for generation of one or more machine learning models comprises a plurality of datasets, wherein each dataset of the plurality of datasets is associated with a particular medical procedure of the plurality of medical procedures, wherein the program instructions further cause the at least one processor to: train the one or more machine learning models based on a first dataset of the plurality of datasets to provide one or more trained machine learning models; assign a unique identifier to the first dataset based on training the one or more machine learning models; assign the unique identifier to the one or more trained machine learning models; and validate an output of the one or more trained machine learning models based on the data associated with the regulatory guidance and approval process, wherein the program instructions that cause the at least one processor to validate the output of the one or more trained machine learning models cause the at least one processor to: validate the first dataset and the one or more trained machine learning models based on the unique identifier assigned to the first dataset and the one or more trained machine learning models.

Clause 27: The computer program product of any of clauses 19-26, wherein the program instructions that cause the at least one processor to deploy the one or more machine learning models to the production environment cause the at least one processor to: deploy the one or more machine learning models to at least one of the following: a production environment of a third-party platform that is not associated with the AI medical device platform; a production environment of the AI medical device platform; a platform that includes a standardized container; or any combination thereof.

Clause 28: A system for generation of artificial intelligence (AI) based automated healthcare applications, comprising: a data repository system, wherein the data repository system stores at least one of the following: formatted data associated with a plurality of medical procedures for generation of one or more non-machine learning automated healthcare applications, data associated with a regulatory guidance and approval process, or any combination thereof; an AI medical device platform that comprises at least one processor in communication with the data repository system, the at least one processor configured to: receive the formatted data associated with a plurality of medical procedures for generation of one or more non-machine learning automated healthcare applications; receive the data associated with one or more regulatory guidance and approval processes; generate one or more non-machine learning automated healthcare applications configured to predict one or more aspects associated with a medical procedure based on the formatted data associated with the plurality of medical procedures and the data associated with the regulatory guidance and approval process; deploy the one or more non-machine learning automated healthcare applications to a production environment based on generating the one or more non-machine learning automated healthcare applications.

Clause 29: The system of clause 28, wherein the data repository system comprises: a data ingestion subsystem to receive data from a plurality of data sources; a data storage subsystem to store data for the data repository system; a data processing subsystem to transform the data of the plurality of data sources based on extract, transform, load (etl) pipelines to provide transformed data to be used for generation of the one or more non-machine learning automated healthcare applications; a data analytics subsystem to provide access to data visualization tools and database analytics tools to be used for generation of the one or more non-machine learning automated healthcare applications; and a data cloud foundation subsystem to provide one or more rules associated with control operations for the data repository system.

Clause 30: The system of clauses 28 or 29, wherein the data repository system is configured to: receive initial data associated with one or more data records from a plurality of data sources, wherein the plurality of data sources comprises at least one of the following: an electronic medical record (EMR) system; a medical imaging system; a fluid injection system; a pathology information system; a laboratory information system; or any combination thereof; and perform one or more data curation procedures on the initial data associated with one or more data records to provide the formatted data associated with a plurality of medical procedures for generation of one or more non-machine learning automated healthcare applications.

Clause 31: The system of any of clauses 28-30, wherein, when generating one or more non-machine learning automated healthcare applications configured to predict one or more aspects associated with a medical procedure, the at least one processor is configured to: generate the one or more non-machine learning automated healthcare applications based on the formatted data associated with a plurality of medical procedures for generation of one or more non-machine learning automated healthcare applications to provide one or more generated non-machine learning automated healthcare applications; and validate an output of the one or more generated non-machine learning automated healthcare applications based on the data associated with the regulatory guidance and approval process.

Clause 32: The system of any of clauses 28-31, wherein the at least one processor is further configured to: receive a request for access to a particular machine learning model of a plurality of non-machine learning automated healthcare applications; determine whether the request for access complies with one or more criteria associated with access to the particular machine learning model; and provide access to the particular machine learning model based on determining that the request for access complies with one or more criteria associated with access to the particular machine learning model.

Clause 33: The system of any of clauses 28-32, wherein the formatted data associated with a plurality of medical procedures for generation of one or more non-machine learning automated healthcare applications comprises a plurality of datasets, wherein each dataset of the plurality of datasets is associated with a particular medical procedure of the plurality of medical procedures, wherein the at least one processor is further configured to: receive a request for access to a particular dataset of a plurality of datasets; determine whether the request for access complies with one or more criteria associated with access to the particular dataset; and provide access to the particular dataset based on determining that the request for access complies with one or more criteria associated with access to the particular dataset.

Clause 34: The system of any of clauses 28-33, wherein, when providing access to the particular dataset, the at least one processor is configured to: perform an application programming interface (API) call associated with the particular dataset to the data repository system to provide access to the particular dataset.

Clause 35: The system of any of clauses 28-34, wherein the formatted data associated with a plurality of medical procedures for generation of one or more non-machine learning automated healthcare applications comprises a plurality of datasets, wherein each dataset of the plurality of datasets is associated with a particular medical procedure of the plurality of medical procedures, wherein the at least one processor is further configured to: generate the one or more non-machine learning automated healthcare applications based on a first dataset of the plurality of datasets to provide one or more generated non-machine learning automated healthcare applications; assign a unique identifier to the first dataset based on generating the one or more non-machine learning automated healthcare applications; assign the unique identifier to the one or more generated non-machine learning automated healthcare applications; and validate an output of the one or more generated non-machine learning automated healthcare applications based on the data associated with the regulatory guidance and approval process, wherein when validating the output of the one or more generated non-machine learning automated healthcare applications, the at least one processor is configured to: validate the first dataset and the one or more generated non-machine learning automated healthcare applications based on the unique identifier assigned to the first dataset and the one or more generated non-machine learning automated healthcare applications.

Clause 36: The system of any of clauses 28-35, wherein, when deploying the one or more non-machine learning automated healthcare applications to the production environment, the at least one processor is configured to: deploy the one or more non-machine learning automated healthcare applications to at least one of the following: a production environment of a third-party platform that is not associated with the AI medical device platform; a production environment of the AI medical device platform; a platform that includes a standardized container; or any combination thereof.

Clause 37: A method for generation of artificial intelligence (AI) based automated healthcare applications, comprising: receiving, with at least one processor of an AI medical device platform, formatted data associated with a plurality of medical procedures for generation of one or more non-machine learning automated healthcare applications from a data repository system; receiving, with the at least one processor, data associated with one or more regulatory guidance and approval processes; generating, with the at least one processor, one or more non-machine learning automated healthcare applications configured to predict one or more aspects associated with a medical procedure based on the formatted data associated with a plurality of medical procedures for generation of one or more non-machine learning automated healthcare applications and the data associated with the regulatory guidance and approval process; and deploying, with the at least one processor, the one or more non-machine learning automated healthcare applications to a production environment based on generating the one or more non-machine learning automated healthcare applications.

Clause 38: The method of clause 37, further comprising: receiving initial data associated with one or more data records from a plurality of data sources, wherein the plurality of data sources comprises at least one of the following: an electronic medical record (EMR) system; a medical imaging system; a fluid injection system; a pathology information system; a laboratory information system; any combination thereof.

Clause 39: The method of clauses 37 or 38, further comprising: performing one or more data curation procedures on the initial data associated with one or more data records to provide the formatted data associated with a plurality of medical procedures for generation of one or more non-machine learning automated healthcare applications.

Clause 40: The method of any of clauses 37-39, wherein generating one or more non-machine learning automated healthcare applications configured to predict one or more aspects associated with a medical procedure comprises: generating the one or more non-machine learning automated healthcare applications based on the formatted data associated with the plurality of medical procedures to provide one or more generated non-machine learning automated healthcare applications; and validating an output of the one or more generated non-machine learning automated healthcare applications based on the data associated with the regulatory guidance and approval process.

Clause 41: The method of any of clauses 37-40, further comprising: receiving a request for access to a particular machine learning model of a plurality of non-machine learning automated healthcare applications; determining whether the request for access complies with one or more criteria associated with access to the particular machine learning model; and providing access to the particular machine learning model based on determining that the request for access complies with one or more criteria associated with access to the particular machine learning model.

Clause 42: The method of any of clauses 37-41, wherein the formatted data associated with a plurality of medical procedures for generation of one or more non-machine learning automated healthcare applications comprises a plurality of datasets, wherein each dataset of the plurality of datasets is associated with a particular medical procedure of the plurality of medical procedures, and wherein the method further comprises: receiving a request for access to a particular dataset of a plurality of datasets; determining whether the request for access complies with one or more criteria associated with access to the particular dataset; and providing access to the particular dataset based on determining that the request for access complies with one or more criteria associated with access to the particular dataset.

Clause 43: The method of any of clauses 37-42, wherein providing access to the particular dataset comprises: performing an application programming interface (API) call associated with the particular dataset to the data repository system to provide access to the particular dataset.

Clause 44: The method of any of clauses 37-43, wherein the formatted data associated with a plurality of medical procedures for generation of one or more non-machine learning automated healthcare applications comprises a plurality of datasets, wherein each dataset of the plurality of datasets is associated with a particular medical procedure of the plurality of medical procedures, and wherein the method further comprises: generating the one or more non-machine learning automated healthcare applications based on a first dataset of the plurality of datasets to provide one or more generated non-machine learning automated healthcare applications; assigning a unique identifier to the first dataset based on generating the one or more non-machine learning automated healthcare applications; assigning the unique identifier to the one or more generated non-machine learning automated healthcare applications; validating an output of the one or more generated non-machine learning automated healthcare applications based on the data associated with the regulatory guidance and approval process, wherein validating the output of the one or more generated non-machine learning automated healthcare applications comprises: validating the first dataset and the one or more generated non-machine learning automated healthcare applications based on the unique identifier assigned to the first dataset and the one or more generated non-machine learning automated healthcare applications.

Clause 45: The method of any of clauses 37-44, wherein deploying the one or more non-machine learning automated healthcare applications to the production environment comprises: deploying the one or more non-machine learning automated healthcare applications to at least one of the following: a production environment of a third-party platform that is not associated with the AI medical device platform; a production environment of the AI medical device platform; a platform that includes a standardized container; or any combination thereof.

Clause 46: A computer program product for generation of artificial intelligence (AI) based automated healthcare applications comprising at least one non-transitory computer-readable medium including program instructions that, when executed by at least one processor, cause the at least one processor to: receive formatted data associated with a plurality of medical procedures for generation of one or more non-machine learning automated healthcare applications from a data repository system; receive data associated with one or more regulatory guidance and approval processes from the data repository system; generate one or more non-machine learning automated healthcare applications configured to predict one or more aspects associated with a medical procedure based on the formatted data associated with the plurality of medical procedures and the data associated with the regulatory guidance and approval process; and deploy the one or more non-machine learning automated healthcare applications to a production environment based on generating the one or more non-machine learning automated healthcare applications.

Clause 47: The computer program product of clause 46, wherein the program instructions further cause the at least one processor to: receive initial data associated with one or more data records from a plurality of data sources, wherein the plurality of data sources comprises at least one of the following: an electronic medical record (EMR) system; a medical imaging system; a fluid injection system; a pathology information system; a laboratory information system; any combination thereof.

Clause 48: The computer program product of clauses 46 or 47, wherein the program instructions further cause the at least one processor to: perform one or more data curation procedures on the initial data associated with one or more data records to provide the formatted data associated with a plurality of medical procedures for generation of one or more non-machine learning automated healthcare applications.

Clause 49: The computer program product of any of clauses 46-48, wherein the program instructions that cause the at least one processor to generate one or more non-machine learning automated healthcare applications configured to predict one or more aspects associated with a medical procedure cause the at least one processor to: train the one or more non-machine learning automated healthcare applications based on the formatted data associated with a plurality of medical procedures for generation of one or more non-machine learning automated healthcare applications to provide one or more generated non-machine learning automated healthcare applications; and validate an output of the one or more generated non-machine learning automated healthcare applications based on the data associated with the regulatory guidance and approval process.

Clause 50: The computer program product of any of clauses 46-49, wherein the program instructions further cause the at least one processor to: receive a request for access to a particular machine learning model of a plurality of non-machine learning automated healthcare applications; determine whether the request for access complies with one or more criteria associated with access to the particular machine learning model; and provide access to the particular machine learning model based on determining that the request for access complies with one or more criteria associated with access to the particular machine learning model.

Clause 51: The computer program product of any of clauses 46-50, wherein the formatted data associated with a plurality of medical procedures for generation of one or more non-machine learning automated healthcare applications comprises a plurality of datasets, wherein each dataset of the plurality of datasets is associated with a particular medical procedure of the plurality of medical procedures, wherein the program instructions further cause the at least one processor to: receive a request for access to a particular dataset of a plurality of datasets; determine whether the request for access complies with one or more criteria associated with access to the particular dataset; and provide access to the particular dataset based on determining that the request for access complies with one or more criteria associated with access to the particular dataset.

Clause 52: The computer program product of any of clauses 46-51, wherein the program instructions that cause the at least one processor to provide access to the particular dataset cause the at least one processor to: perform an application programming interface (API) call associated with the particular dataset to the data repository system to provide access to the particular dataset.

Clause 53: The computer program product of any of clauses 46-52, wherein the formatted data associated with a plurality of medical procedures for generation of one or more non-machine learning automated healthcare applications comprises a plurality of datasets, wherein each dataset of the plurality of datasets is associated with a particular medical procedure of the plurality of medical procedures, wherein the program instructions further cause the at least one processor to: train the one or more non-machine learning automated healthcare applications based on a first dataset of the plurality of datasets to provide one or more generated non-machine learning automated healthcare applications; assign a unique identifier to the first dataset based on generating the one or more non-machine learning automated healthcare applications; assign the unique identifier to the one or more generated non-machine learning automated healthcare applications; and validate an output of the one or more generated non-machine learning automated healthcare applications based on the data associated with the regulatory guidance and approval process, wherein the program instructions that cause the at least one processor to validate the output of the one or more generated non-machine learning automated healthcare applications cause the at least one processor to: validate the first dataset and the one or more generated non-machine learning automated healthcare applications based on the unique identifier assigned to the first dataset and the one or more generated non-machine learning automated healthcare applications.

Clause 54: The computer program product of any of clauses 46-53, wherein the program instructions that cause the at least one processor to deploy the one or more non-machine learning automated healthcare applications to the production environment cause the at least one processor to: deploy the one or more non-machine learning automated healthcare applications to at least one of the following: a production environment of a third-party platform that is not associated with the AI medical device platform; a production environment of the AI medical device platform; a platform that includes a standardized container; or any combination thereof.

These and other features and characteristics of the present disclosure, as well as the methods of operation and functions of the related elements of structures and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the present disclosure. As used in the specification and the claims, the singular form of “a,” “an,” and “the” include plural referents unless the context clearly dictates otherwise.

### BRIEF DESCRIPTION OF THE DRAWINGS

Additional advantages and details of non-limiting embodiments are explained in greater detail below with reference to the exemplary embodiments that are illustrated in the accompanying schematic figures, in which:

FIG. 1A is a diagram of a non-limiting embodiment of an environment in which systems, methods, and/or computer program products described herein, may be implemented according to the present disclosure;

FIG. 1B is a diagram of a non-limiting embodiment of a system for generation of artificial intelligence (AI) based automated healthcare applications;

FIG. 2 is a diagram of a non-limiting embodiment of components of one or more devices and/or one or more systems of FIGS. 1A and 1B;

FIG. 3 is a flowchart of a non-limiting embodiment of a process for generation of AI based automated healthcare applications using an AI medical device platform;

FIG. 4 is a diagram of a non-limiting embodiment of an AI medical device platform;

FIG. 5 is a diagram of a non-limiting embodiment of a data repository system; and

FIGS. 6A-6D are diagrams of a non-limiting embodiment of an implementation of a process for generation of AI based automated healthcare applications using an AI medical device platform.

### DESCRIPTION

For purposes of the description hereinafter, the terms “end,” “upper,” “lower,” “right,” “left,” “vertical,” “horizontal,” “top,” “bottom,” “lateral,” “longitudinal,” and derivatives thereof shall relate to the disclosure as it is oriented in the drawing figures. However, it is to be understood that the disclosure may assume various alternative variations and step sequences, except where expressly specified to the contrary. It is also to be understood that the specific devices and processes illustrated in the attached drawings, and described in the following specification, are simply exemplary embodiments of the disclosure. Hence, specific dimensions and other physical characteristics related to the embodiments or aspects disclosed herein are not to be considered as limiting unless otherwise indicated.

No aspect, component, element, structure, act, step, function, instruction, and/or the like used herein should be construed as critical or essential unless explicitly described as such. Also, as used herein, the articles “a” and “an” are intended to include one or more items and may be used interchangeably with “one or more” and “at least one.” Furthermore, as used herein, the term “set” is intended to include one or more items (e.g., related items, unrelated items, a combination of related and unrelated items, and/or the like) and may be used interchangeably with “one or more” or “at least one.” Where only one item is intended, the term “one” or similar language is used. Also, as used herein, the terms “has,” “have,” “having,” or the like are intended to be open-ended terms. Further, the phrase “based on” is intended to mean “based at least partially on” unless explicitly stated otherwise. In addition, reference to an action being “based on” a condition may refer to the action being “in response to” the condition. For example, the phrases “based on” and “in response to” may, in some non-limiting embodiments or aspects, refer to a condition for automatically triggering an action (e.g., a specific operation of an electronic device, such as a computing device, a processor, and/or the like).

As used herein, the terms “communication” and “communicate” may refer to the reception, receipt, transmission, transfer, provision, and/or the like of information (e.g., data, signals, messages, instructions, commands, and/or the like). For one unit (e.g., a device, a system, a component of a device or system, combinations thereof, and/or the like) to be in communication with another unit means that the one unit is able to directly or indirectly receive information from and/or transmit information to the other unit. This may refer to a direct or indirect connection that is wired and/or wireless in nature. Additionally, two units may be in communication with each other even though the information transmitted may be modified, processed, relayed, and/or routed between the first and second unit. For example, a first unit may be in communication with a second unit even though the first unit passively receives information and does not actively send information to the second unit. As another example, a first unit may be in communication with a second unit if at least one intermediary unit (e.g., a third unit located between the first unit and the second unit) processes information received from the first unit and sends the processed information to the second unit. In some non-limiting embodiments, information may refer to a network packet (e.g., a data packet and/or the like) that includes data.

Some non-limiting embodiments or aspects may be described herein in connection with thresholds. As used herein, satisfying a threshold may refer to a value being greater than the threshold, more than the threshold, higher than the threshold, greater than or equal to the threshold, less than the threshold, fewer than the threshold, lower than the threshold, less than or equal to the threshold, equal to the threshold, etc.

Systems, methods, and computer program products are disclosed that provide solutions to the above mentioned challenges. For example, as disclosed herein, a system for generation of artificial intelligence (AI) based automated healthcare applications may include a data repository system and an AI medical device platform. In some non-limiting embodiments, the data repository system may store at least one of the following: formatted data associated with a plurality of medical procedures for generation of one or more machine learning models, data associated with a regulatory guidance and approval process, or any combination thereof. In some non-limiting embodiments, the AI medical device platform may include at least one processor in communication with the data repository system, the at least one processor configured to receive the formatted data associated with a plurality of medical procedures for generation of one or more machine learning models, receive the data associated with one or more regulatory guidance and approval processes, generate one or more machine learning models configured to predict one or more aspects associated with a medical procedure based on the data associated with the plurality of medical procedures and the data associated with the regulatory guidance and approval process, and deploy the one or more machine learning models to a production environment based on generating the one or more machine learning models.

In some non-limiting embodiments, the data repository system includes a data ingestion subsystem to receive data from a plurality of data sources, a data storage subsystem to store data for the data repository system, a data processing subsystem to transform the data of the plurality of data sources based on extract, transform, load (etl) pipelines to provide transformed data to be used for generation of the one or more machine learning models, a data analytics subsystem to provide access to data visualization tools and database analytics tools to be used for generation of the one or more machine learning models, and a data cloud foundation subsystem to provide one or more rules associated with control operations for the data repository system.

In some non-limiting embodiments, the data repository system may be configured to receive initial data associated with one or more data records from a plurality of data sources, wherein the plurality of data sources comprises at least one of the following: an electronic medical record (EMR) system, a medical imaging system, a fluid injection system, a pathology information system, a laboratory information system, or any combination thereof; and perform one or more data curation procedures on the initial data associated with one or more data records to provide the formatted data associated with a plurality of medical procedures for generation of one or more machine learning models.

In some non-limiting embodiments, when generating one or more machine learning models configured to predict one or more aspects associated with a medical procedure, the at least one processor may be configured to train the one or more machine learning models based on the formatted data associated with a plurality of medical procedures for generation of one or more machine learning models to provide one or more trained machine learning models, and validate an output of the one or more trained machine learning models based on the data associated with the regulatory guidance and approval process.

In some non-limiting embodiments, the at least one processor may be further configured to receive a request for access to a particular machine learning model of a plurality of machine learning models, determine whether the request for access complies with one or more criteria associated with access to the particular machine learning model, and provide access to the particular machine learning model based on determining that the request for access complies with one or more criteria associated with access to the particular machine learning model. In some non-limiting embodiments, the formatted data associated with a plurality of medical procedures for generation of one or more machine learning models may include a plurality of datasets, and each dataset of the plurality of datasets may be associated with a particular medical procedure of the plurality of medical procedures, and the at least one processor may be further configured to receive a request for access to a particular dataset of a plurality of datasets, determine whether the request for access complies with one or more criteria associated with access to the particular dataset, and provide access to the particular dataset based on determining that the request for access complies with one or more criteria associated with access to the particular dataset.

In some non-limiting embodiments, when providing access to the particular dataset, the at least one processor may be configured to perform an application programming interface (API) call associated with the particular dataset to the data repository system to provide access to the particular dataset.

In some non-limiting embodiments, the formatted data associated with a plurality of medical procedures for generation of one or more machine learning models may include a plurality of datasets, and each dataset of the plurality of datasets is associated with a particular medical procedure of the plurality of medical procedures, and the at least one processor may be further configured to train the one or more machine learning models based on a first dataset of the plurality of datasets to provide one or more trained machine learning models, assign a unique identifier to the first dataset based on training the one or more machine learning models, assign the unique identifier to the one or more trained machine learning models, and validate an output of the one or more trained machine learning models based on the data associated with the regulatory guidance and approval process. In some non-limiting embodiments, when validating the output of the one or more trained machine learning models, the at least one processor may be configured to validate the first dataset and the one or more trained machine learning models based on the unique identifier assigned to the first dataset and the one or more trained machine learning models.

In some non-limiting embodiments, when deploying the one or more machine learning models to the production environment, the at least one processor may be configured to deploy the one or more machine learning models to at least one of the following: a production environment of a third-party platform that is not associated with the AI medical device platform, a production environment of the AI medical device platform, a platform that includes a standardized container, or any combination thereof.

In this way, the present disclosure provides for efficiently developing and/or deploying automated healthcare applications, including AI based SaMDs (e.g., automated healthcare applications that include one or more machine learning models). Additionally, the present disclosure provides an AI medical device platform and a data repository system that allows for a plurality of users (e.g., unrelated users) to access large amounts of data associated with plurality of medical procedures, as well as data associated with one or more regulatory guidance and approval processes for generation of automated healthcare applications, which provides for determining whether the automated healthcare applications meet regulatory standards for regulatory guidance and approval, prior to deployment of the automated healthcare applications.

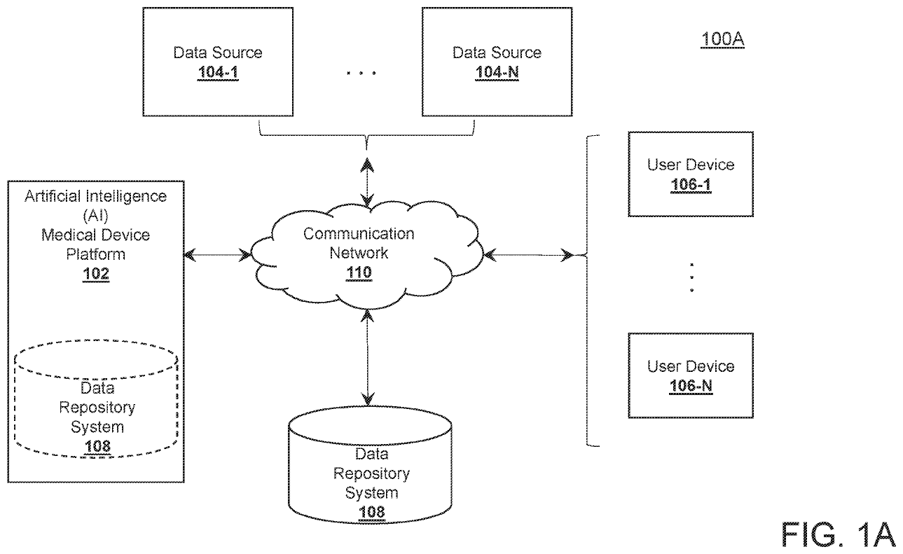

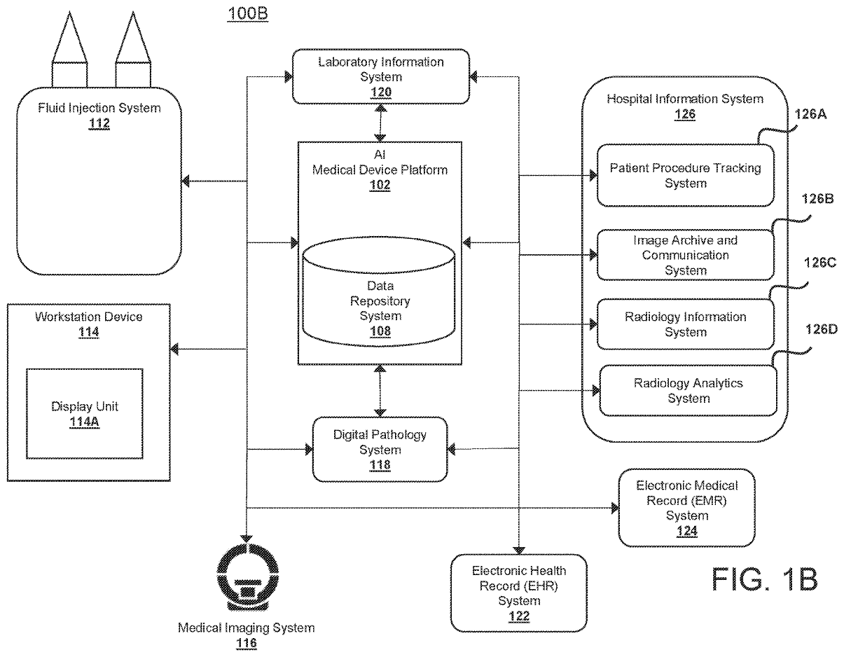

Referring now to FIG. 1A, FIG. 1A is a diagram of a non-limiting embodiment of an environment 100A in which devices, systems, methods, and/or computer program products, described herein, may be implemented. As shown in FIG. 1A, environment 100A includes artificial intelligence (AI) medical device platform 102, data sources 104-1 through 104-N (referred to hereafter individually as data source 104, or together as data sources 104, where appropriate), user devices 106-1 through 106-N (referred to hereafter individually as user device 106, or together as user devices 106, where appropriate), and data repository system 108.

AI medical device platform 102 may interconnect (e.g., establish a connection to communicate with and/or the like) with data sources 104, user devices 106, and/or data repository system 108 via communication network 110. In some non-limiting embodiments, AI medical device platform 102 may interconnect (e.g., establish a connection to communicate with and/or the like) with data sources 104, user devices 106, and/or data repository system 108 via wired connections, wireless connections, or a combination of wired and wireless connections.

In some non-limiting embodiments, AI medical device platform 102 may include one or more devices capable of being in communication with data sources 104, user devices 106, and/or data repository system 108 via communication network 110. For example, AI medical device platform 102 may include a server, a group of servers, such as a cloud-based solution that includes a plurality of servers (e.g., a private cloud solution, a public cloud, a hybrid cloud, a multi-cloud, etc.), and/or other like devices. Additionally or alternatively, AI medical device platform 102 may include a computing device, such as a desktop computer, a mobile device (e.g., a tablet, a smartphone, a wearable device, etc.), and/or the like. In some non-limiting embodiments, AI medical device platform 102 may include one or more (e.g., a plurality of) applications (e.g., software applications) that perform a set of functionalities on an external application programming interface (API) to send data to an external system, such as user device 106 or data repository system 108, associated with the external API and to receive data from the external system associated with the external API. In some non-limiting embodiments, AI medical device platform 102 may include one or more subsystems. For example, AI medical device platform 102 may include one or more subsystems that pertain to generation of one or more machine learning models (e.g., one or more automated healthcare applications that include one or more machine learning models). In some non-limiting embodiments, AI medical device platform 102 may include data repository system 108.

In some non-limiting embodiments, AI medical device platform 102 may generate (e.g., train, validate, re-train, and/or the like), store, and/or implement (e.g., operate, provide inputs to and/or outputs from, and/or the like) one or more machine learning models. For example, AI medical device platform 102 may generate one or more machine learning models by fitting (e.g., validating, testing, etc.) one or more machine learning models against data used for training (e.g., training data). In some non-limiting embodiments or aspects, AI medical device platform 102 may generate, store, and/or implement one or more machine learning models that are provided for a production environment (e.g., a runtime environment, a real-time environment, an environment where software applications and/or services are deployed and made available to end users, etc.) used for providing inferences (e.g., secure inferences) based on data inputs in a live situation (e.g., real-time situation) in a production environment. In some non-limiting embodiments, a production environment may include a target information technology (IT) setup comprising hardware and/or software executed on hardware for reliability and redundancy. Additionally or alternatively, AI medical device platform 102 may generate, store, and/or implement one or more machine learning models that are provided for a non-production environment (e.g., an offline environment, a training environment, etc.) used for providing inferences based on data inputs in a situation that is not live. In some non-limiting embodiments or aspects, AI medical device platform 102 may be in communication with a data storage device (data repository system 108), which may be local or remote to AI medical device platform 102.

In some non-limiting embodiments, AI medical device platform 102 may provide platform level services that include services for isolated software instances, user interfaces for interaction, services associated with a marketplace for all aspects of automated healthcare applications, services associated with contracting for use of AI medical device platform 102, services for consumption monitoring and/or billing associated with use of AI medical device platform 102, services for onboarding users of AI medical device platform 102, procurement services related to AI medical device platform 102, and/or user management services (e.g., authentication, authorization, levels of access, etc.) related to AI medical device platform 102.

In some non-limiting embodiments, data source 104 may include one or more devices capable of being in communication with AI medical device platform 102, user device 106, and data repository system 108 via communication network 110 and be capable of performing fluid injection procedures. For example, data source 104 may include a server, a computing device, such as a desktop computer, a mobile device (e.g., a tablet, a smartphone, a wearable, such as a wearable health sensor, etc.), and/or the like. In some non-limiting embodiments, data source 104 may include a hospital information system, a picture archive and communication system (PACS), an EMR system, a medical imaging system (e.g., an imaging scanner), a fluid injection system (e.g., a fluid injector), a device associated with a facility, such as a communication device associated with a medical device (e.g., a hand-held medical device, a wearable medical device, such as a portable health sensor, etc.), a fluid injection system, a pathology information system, a laboratory information system, and/or a device associated with a patient (e.g., a user device, such as a computing device operated by a patient). Additionally or alternatively, data source 104 may include a reporting system (e.g., a system that generates medical reports based on healthcare data), a device associated with a facility (e.g., a hospital), and/or a device associated with a patient.

In some non-limiting embodiments, user device 106 may include one or more devices capable of being in communication with AI medical device platform 102, data sources 104, and/or data repository system 108 via communication network 110. For example, user device 106 may include a server, a group of servers, a computing device, such as a desktop computer, a mobile device (e.g., a tablet, a smartphone, a wearable device, etc.), and/or the like. In some non-limiting embodiments or aspects, user device 106 may be associated with a user (e.g., an individual operating user device 106).

In some non-limiting embodiments, data repository system 108 may include one or more devices capable of being in communication with AI medical device platform 102, data sources 104, and/or user device 106 via communication network 110. For example, data repository system 108 may include a server, a group of servers, a cloud platform, and/or the like. In some non-limiting embodiments, data repository system 108 may include a data lake for storing, processing, and/or securing large amounts of data, such as data records (e.g., electronic medical records of patients, electronic health records of patients, etc.). In some examples, data repository system 108 may receive, store, process, and/or secure a plurality of data records, such as at least 1,000 data records, 10,000 data records, 100,000,000 data records, 1,000,000,000 data records, 1,000,000,000,000 data records, and/or the like. In another example, data repository system 108 may include one or more one or more machine learning models that are each stored on a device (e.g., a server).

In some non-limiting embodiments, data repository system 108 may include a plurality of subsystems. In some non-limiting embodiments, one or more of the plurality of subsystems of data repository system 108 may perform a set of functionalities on an external API to send data to an external system, such as AI medical device platform 102 or another data repository, associated with the external API, and/or to receive data from AI medical device platform 102 and/or the other data repository associated with the external API.

In some non-limiting embodiments, communication network 110 may include one or more wired and/or wireless networks. For example, communication network 110 may include a cellular network (e.g., a long-term evolution (LTE) network, a third generation (3G) network, a fourth generation (4G) network, a fifth generation (5G) network, a code division multiple access (CDMA) network, and/or the like), a local area network (LAN), a wide area network (WAN), a wireless LAN (WLAN), a private network, an ad hoc network, an intranet, the Internet, a fiber optic-based network, an Ethernet network, a universal serial bus (USB) network, a cloud computing network, and/or the like, and/or a combination of some or all of these or other types of networks.

The number and arrangement of systems and/or devices shown in FIG. 1A are provided as an example. There may be additional systems and/or devices, fewer systems and/or devices, different systems and/or devices, or differently arranged systems and/or devices than those shown in FIG. 1A. Furthermore, two or more systems and/or devices shown in FIG. 1A may be implemented within a single system or a single device, or a single system or a single device shown in FIG. 1A may be implemented as multiple, distributed systems or devices. Additionally or alternatively, a set of systems or a set of devices (e.g., one or more systems, one or more devices) of environment 100A may perform one or more functions described as being performed by another set of systems or another set of devices of environment 100A.

As shown in FIG. 1B, system 100B includes AI medical device platform 102, which includes data repository system 108, fluid injection system 112, workstation device 114, which includes display unit 114A, medical imaging system 116, digital pathology system 118, laboratory information system 120, electronic health record (EHR) system 122, electronic medical record (EMR) system 124, and hospital information system 126. In some non-limiting embodiments, AI medical device platform 102 (e.g., and data repository system 108) may interconnect (e.g., establish a connection to communicate with and/or the like) fluid injection system 112, workstation device 114, medical imaging system 116, digital pathology system 118, laboratory information system 120, EHR system 122, EMR system 124, and hospital information system 126 via wired connections, wireless connections, or a combination of wired and wireless connections. In some non-limiting embodiments, fluid injection system 112, workstation device 114, which includes display unit 114A, medical imaging system 116, digital pathology system 118, laboratory information system 120, electronic health record (EHR) system 122, electronic medical record (EMR) system 124, and/or hospital information system 126 may be the same as or similar to data sources 104.

In some non-limiting embodiments, fluid injection system 112 may include one or more devices capable of being in communication with AI medical device platform 102, workstation device 114, medical imaging system 116, digital pathology system 118, laboratory information system 120, EHR system 122, EMR system 124, and/or hospital information system 126 via a communication network (e.g., communication network 110). For example, fluid injection system 112 may include one or more computing devices, such as one or more computers, one or more servers (e.g., a cloud server, a group of servers, etc.), one or more desktop computers, one or mobile devices (e.g., one or more tablets, one or more smartphones, etc.), and/or the like. In some non-limiting embodiments, fluid injection system 112 may include one or more injection devices (e.g., one or more fluid injection devices, one or more fluid injectors). In some non-limiting embodiments, fluid injection system 112 is configured to administer (e.g., inject, deliver, etc.) contrast fluid including a contrast agent to a patient, and/or administer an aqueous fluid, such as saline, to a patient before, during, and/or after administering the contrast fluid. For example, fluid injection system 112 can inject one or more prescribed dosages of contrast fluid directly into a patient's blood stream via a hypodermic needle and syringe. In some non-limiting embodiments, fluid injection system 112 may be configured to continually administer the aqueous fluid to a patient through a peripheral intravenous line (PIV) and catheter, and one or more prescribed dosages of contrast fluid may be introduced into the PIV and administered via the catheter to the patient. In some non-limiting embodiments, fluid injection system 112 is configured to inject a dose of contrast fluid followed by administration of a particular volume of the aqueous fluid. In some non-limiting embodiments, fluid injection system 112 may include one or more exemplary fluid injection devices that are disclosed in: U.S. patent application Ser. No. 09/715,330, filed on Nov. 17, 2000, issued as U.S. Pat. No. 6,643,537; U.S. patent application Ser. No. 09/982,518, filed on Oct. 18, 2001, issued as U.S. Pat. No. 7,094,216; U.S. patent application Ser. No. 10/825,866, filed on Apr. 16, 2004, issued as U.S. Pat. No. 7,556,619; U.S. patent application Ser. No. 12/437,011, filed May 7, 2009, issued as U.S. Pat. No. 8,337,456; U.S. patent application Ser. No. 12/476,513, filed Jun. 2, 2009, issued as U.S. Pat. No. 8,147,464; and U.S. patent application Ser. No. 11/004,670, filed on Dec. 3, 2004, issued as U.S. 8,540,698, the disclosures of each of which are incorporated herein by reference in their entireties. In some non-limiting embodiments, fluid injection system 112 may include the MEDRAD® Stellant CT Injection System, the MEDRAD® Stellant FLEX CT Injection System, the MEDRAD® MRXperion MR Injection System, the MEDRAD® Mark 7 Arterion Injection System, the MEDRAD® Intego PET Infusion System, or the MEDRAD® Centargo CT Injection System, all of which are provided by Bayer Healthcare LLC.

In some non-limiting embodiments, workstation device 114 includes one or more devices capable of being in communication with AI medical device platform 102, fluid injection system 112, medical imaging system 116, digital pathology system 118, laboratory information system 120, EHR system 122, EMR system 124, and/or hospital information system 126 via a communication network (e.g., communication network 110). For example, workstation device 114 may include a computing device, such as one or more computers, including a desktop computer, a laptop, a tablet, and/or the like. In some non-limiting embodiments, workstation device 114 may provide a control interface for controlling operation of fluid injection system 112, including providing inputs to fluid injection system 112. Additionally or alternatively, workstation device 114 may display operational parameters of fluid injection system 112 during operation (e.g., during real-time operation) of fluid injection system 112. In some non-limiting embodiments, workstation device 114 may provide interconnectivity between fluid injection system 112 and other devices or systems, such as medical imaging system 116. In some non-limiting embodiments, workstation device 114 may include the Certegra® Workstation provided by Bayer.

In some non-limiting embodiments, medical imaging system 116 may include one or more devices capable of being in communication with AI medical device platform 102, fluid injection system 112, workstation device 114, digital pathology system 118, laboratory information system 120, EHR system 122, EMR system 124, and/or hospital information system 126 via a communication network (e.g., communication network 110). In some non-limiting embodiments, medical imaging system 116 may include an ultrasound system, an echocardiography system, a magnetic resonance imaging (MRI) system, an electromagnetic radiation system, (e.g., a conventional 2-D X-ray, a 3-D computed tomography (CT) scanning system, a fluoroscopy system, etc.), capable of communicating via communication network 110 and capable of performing medical imaging procedures, including medical imaging procedures involving the use of a radiological contrast material. In some non-limiting embodiments, medical imaging system 116 may provide an image of a patient and/or data associated with the image of the patient (e.g., an image of a patient as the result of an imaging study). The data associated with the image of the patient may include data in a Digital Imaging and Communications in Medicine (DICOM) format, which may include metadata, pixel data, and/or additional data associated with an imaging procedure that was performed on the patient to provide the image.

In some non-limiting embodiments, digital pathology system 118 may include one or more devices capable of being in communication with AI medical device platform 102, fluid injection system 112, workstation device 114, medical imaging system 116, laboratory information system 120, EHR system 122, EMR system 124, and/or hospital information system 126 via a communication network (e.g., communication network 110). In some non-limiting embodiments, medical imaging system 116 may include one or more devices that receive, manage, transmit, and/or interpret pathology information including data (e.g., image data, slide data, etc.) analyzed by a microscope, a scanner, and/or other like devices.

In some non-limiting embodiments, laboratory information system 120 may include one or more devices capable of being in communication with AI medical device platform 102, fluid injection system 112, workstation device 114, medical imaging system 116, laboratory information system 120, EHR system 122, EMR system 124, and/or hospital information system 126 via a communication network (e.g., communication network 110). In some non-limiting embodiments, laboratory information system 120 may include one or more devices that record, manage, update, and/or store patient data and/or testing data for clinical and/or anatomic pathology laboratories, including receiving test orders, transmitting orders to laboratory analyzers, tracking orders, results, and/or quality control information, and/or transmitting results to other systems or devices. In some non-limiting embodiments, laboratory information system 120 may include a system designed to support operations of a laboratory (e.g., a medical laboratory). Laboratory information system 120 may include functionality, such as workflow, data tracking, flexible architecture, and/or data exchange interfaces, that supports the use of a laboratory in regulated environments. In some non-limiting embodiments, laboratory information system 120 may include an enterprise resource planning tool that is designed to manage aspects of laboratory informatics (e.g., patient data associated with laboratory processes and/or laboratory testing).

In some non-limiting embodiments, EHR system 122 may include one or more devices capable of being in communication with AI medical device platform 102, fluid injection system 112, workstation device 114, medical imaging system 116, digital pathology system 118, laboratory information system 120, EMR system 124, and/or hospital information system 126 via a communication network (e.g., communication network 110). In some non-limiting embodiments, EHR system 122 may include one or more devices that receive, manage, store, and/or transmit electronic health records that include medical record data associated with a medical record of a patient, such as demographics, medical history, medication and allergies, immunization status, laboratory test results, radiology images, vital signs, personal statistics (e.g., age, weight, height, etc.), billing information, and/or the like, associated with various providers and/or locations (e.g., offices, clinics, hospitals, etc.) of medical care. Additionally or alternatively, EHR system 122 may include a patient portal (e.g., a web-based interface) to allow a patient to interact with a respective electronic medical record for the patient. In some non-limiting embodiments, EMR system 124 may be a data source for EHR system 122.

In some non-limiting embodiments, EMR system 124 may include one or more devices capable of being in communication with AI medical device platform 102, fluid injection system 112, workstation device 114, medical imaging system 116, digital pathology system 118, laboratory information system 120, EHR system 122, and/or hospital information system 126 via a communication network (e.g., communication network 110). In some non-limiting embodiments, EMR system 124 may include one or more devices that receive, manage, store, and/or transmit electronic medical records (e.g., electronic health records), that include medical record data associated with a medical record of a patient, such as demographics, medical history, medication and allergies, immunization status, laboratory test results, radiology images, vital signs, personal statistics (e.g., age, weight, height, etc.), billing information, and/or the like.

In some non-limiting embodiments, hospital information system 126 may include one or more devices capable of being in communication with AI medical device platform 102, fluid injection system 112, workstation device 114, medical imaging system 116, digital pathology system 118, laboratory information system 120, EHR system 122, and/or EMR system 124 via a communication network (e.g., communication network 110). For example, hospital information system 126 may include one or more computing devices, such as one or more desktop computers, one or mobile devices, one or more servers, and/or the like. In some non-limiting embodiments, hospital information system 126 may include one or more subsystems, such as a patient procedure tracking system (e.g., a system that operates a modality worklist, a system that provides patient demographic information for fluid injection procedures and/or medical imaging procedures, etc.), a fluid injector management system, an image archive and communication system (e.g., a picture archive and communication system (PACS)), a radiology information system (RIS), a radiology analytics system (e.g., the Radimetrics® Enterprise Application marketed and sold by Bayer HealthCare LLC), and/or other like systems or devices.

As further shown in FIG. 1B, hospital information system 126 may include a plurality of subsystems. The plurality of subsystems may include patient procedure tracking system 126A, image archive and communication system 126B, radiology information system 126C, and radiology analytics system 126D. In some non-limiting embodiments, AI medical device platform 102 may receive data from hospital information system 126 via a communication network (e.g., communication network 110), according to a communications protocol for communicating the data. For example, AI medical device platform 102 may receive data associated with a patient procedure from hospital information system 126 (e.g., from patient procedure tracking system 126A) via the communication network according to a Digital Imaging and Communications in Medicine (DICOM) communications protocol, data associated with an operation of the fluid injection system from hospital information system 126 via the communication network based on an API call (e.g., an API call from AI medical device platform 102), data associated with a radiology image from hospital information system 126 (e.g., from image archive and communication system 126B) via the communication network according to a DICOM communications protocol, data associated with a patient examination procedure from hospital information system 126 (e.g., from radiology information system 126C) via the communication network according to a Health Level Seven (HL7) standard communications protocol, and/or data associated with radiation dosage during a medical imaging procedure from hospital information system 126 (e.g., from radiology analytics system 126D) via the communication network based on an API call (e.g., an API call from AI medical device platform 102 to radiology analytics system 126D).

In some non-limiting embodiments, AI medical device platform 102 may include a plurality of applications, and each of the plurality of applications may be associated with an API associated with a respective application (e.g., a first API associated with a first application, a second API associated with a second application, a third API associated with a third application, etc.) that allows other systems and/or devices to interface (e.g., communicate, establish a communication interface, etc.) with AI medical device platform 102 and/or that allows AI medical device platform 102 to interface with other systems and/or devices (e.g., individual subsystems of hospital information system 126, such as patient procedure tracking system 126A, image archive and communication system 126B, radiology information system 126C, and/or radiology analytics system 126D). In some non-limiting embodiments, AI medical device platform 102 may provide a user interface (e.g., via an application that includes a user interface, such as a web-based user interface) that allows a user to access information (e.g., such as a medical finding for a patient).

As further shown in FIG. 1B, workstation device 114 may include display unit 114A. In some non-limiting embodiments, display unit 114A may be capable of displaying the user interface (e.g., the web-based user interface) provided by AI medical device platform 102. In some non-limiting embodiments, display unit 114A may include a computing device, such as a smart display unit, a portable computer, such as a tablet, a laptop, and/or the like. In some non-limiting embodiments, display unit 114A may include a touchscreen for receiving inputs by a user. In some non-limiting embodiments, display unit 114A may include a display device (e.g., a monitor, a screen, and/or the like for displaying visual information).

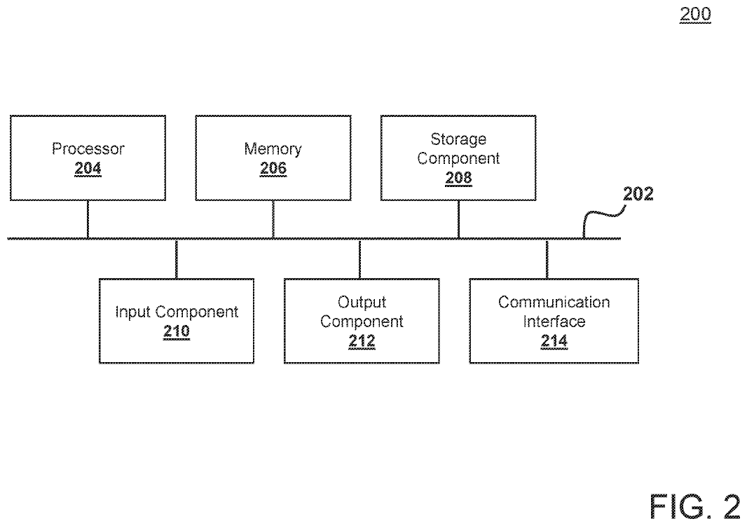

Referring now to FIG. 2, FIG. 2 is a diagram of example components of device 200. Device 200 may correspond to AI medical device platform 102, data source 104, user device 106, data repository system 108, fluid injection system 112, workstation device 114, medical imaging system 116, digital pathology system 118, laboratory information system 120, EHR system 122, EHR system 124, and/or hospital information system 126. In some non-limiting embodiments, AI medical device platform 102, data source 104, user device 106, data repository system 108, fluid injection system 112, workstation device 114, medical imaging system 116, digital pathology system 118, laboratory information system 120, EHR system 122, EHR system 124, and/or hospital information system 126 may include at least one device 200 and/or at least one component of device 200. As shown in FIG. 2, device 200 may include bus 202, processor 204, memory 206, storage component 208, input component 210, output component 212, and communication component 214.

Bus 202 may include a component that permits communication among the components of device 200. In some non-limiting embodiments, processor 204 may be implemented in hardware or a combination of hardware and software. For example, processor 204 may include a processor (e.g., a central processing unit (CPU), a graphics processing unit (GPU), an accelerated processing unit (APU), and/or the like), a microprocessor, a digital signal processor (DSP), and/or any processing component (e.g., a field-programmable gate array (FPGA), an application-specific integrated circuit (ASIC), and/or the like) that can be programmed to perform a function. Memory 206 may include random access memory (RAM), read-only memory (ROM), and/or another type of dynamic or static storage device (e.g., flash memory, magnetic memory, optical memory, and/or the like) that stores information and/or instructions for use by processor 204.

Storage component 208 may store information and/or software related to the operation and use of device 200. For example, storage component 208 may include a hard disk (e.g., a magnetic disk, an optical disk, a magneto-optic disk, a solid state disk, and/or the like), a compact disc (CD), a digital versatile disc (DVD), a floppy disk, a cartridge, a magnetic tape, and/or another type of computer-readable medium, along with a corresponding drive.

Input component 210 may include a component that permits device 200 to receive information, such as via user input (e.g., a touchscreen display, a keyboard, a keypad, a mouse, a button, a switch, a microphone, a camera, and/or the like). Additionally or alternatively, input component 210 may include a sensor for sensing information (e.g., a global positioning system (GPS) component, an accelerometer, a gyroscope, an actuator, and/or the like). Output component 212 may include a component that provides output information from device 200 (e.g., a display, a speaker, one or more light-emitting diodes (LEDs), and/or the like).

Communication component 214 may include a transceiver-like component (e.g., a transceiver, a separate receiver and transmitter, and/or the like) that enables device 200 to communicate with other devices, such as via a wired connection, a wireless connection, or a combination of wired and wireless connections. Communication component 214 may permit device 200 to receive information from another device and/or provide information to another device. For example, communication component 214 may include an Ethernet interface, an optical interface, a coaxial interface, an infrared interface, a radio frequency (RF) interface, a universal serial bus (USB) interface, a Wi-Fi® interface, a cellular network interface, and/or the like.

Device 200 may perform one or more processes described herein. Device 200 may perform these processes based on processor 204 executing software instructions stored by a computer-readable medium, such as memory 206 and/or storage component 208. A computer-readable medium (e.g., a non-transitory computer-readable medium) is defined herein as a non-transitory memory device. A non-transitory memory device includes memory space located inside of a single physical storage device or memory space spread across multiple physical storage devices.

Software instructions may be read into memory 206 and/or storage component 208 from another computer-readable medium or from another device via communication component 214. When executed, software instructions stored in memory 206 and/or storage component 208 may cause processor 204 to perform one or more processes described herein. Additionally or alternatively, hardwired circuitry may be used in place of or in combination with software instructions to perform one or more processes described herein. Thus, embodiments described herein are not limited to any specific combination of hardware circuitry and software.

Memory 206 and/or storage component 208 may include data storage or one or more data structures (e.g., a database and/or the like). Device 200 may be capable of retrieving information from, storing information in, or searching for information stored in the data storage or one or more data structures in memory 206 and/or storage component 208.

The number and arrangement of components shown in FIG. 2 are provided as an example. In some non-limiting embodiments, device 200 may include additional components, fewer components, different components, or differently arranged components than those shown in FIG. 2. Additionally or alternatively, a set of components (e.g., one or more components) of device 200 may perform one or more functions described herein as being performed by another set of components of device 200.

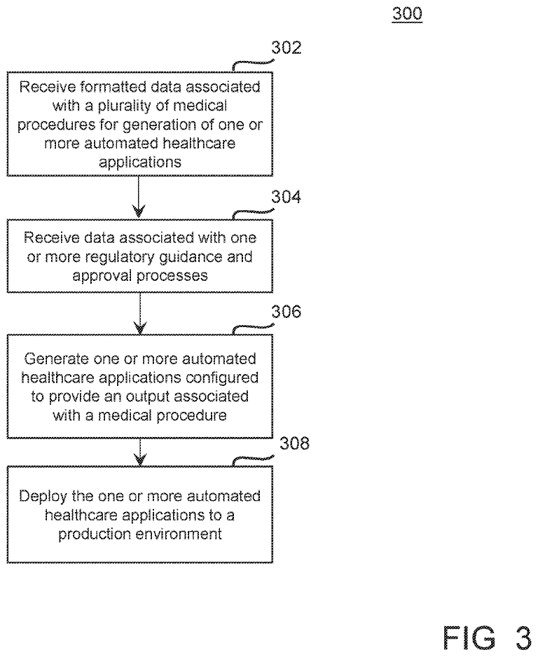

Referring now to FIG. 3, FIG. 3 is a flowchart of a non-limiting embodiment of a process 300 for generation of automated healthcare applications. In some non-limiting embodiments, one or more of the steps of process 300 may be performed (e.g., completely, partially, etc.) by AI medical device platform 102 (e.g., one or more devices of AI medical device platform 102). In some non-limiting embodiments, one or more of the steps of process 300 may be performed (e.g., completely, partially, etc.) by another device or a group of devices separate from or including AI medical device platform 102, data source 104, user device 106, and/or data repository system 108. The steps shown in FIG. 3 are for example purposes only. It will be appreciated that additional, fewer, different, and/or a different order of steps may be used in some non-limiting embodiments or aspects. In some non-limiting embodiments or aspects, a step may be automatically performed in response to performance and/or completion of a prior step. As referred to herein, automated healthcare applications may refer to one or more non-machine learning automated healthcare applications (e.g., one or more automated healthcare applications that are programmed and do not include one or more machine learning models) and/or one or more machine learning automated healthcare applications (e.g., one or more automated healthcare applications that include one or more machine learning models).

As shown in FIG. 3, at step 302, process 300 includes receiving formatted data associated with a plurality of medical procedures for generation of one or more automated healthcare applications. For example, AI medical device platform 102 may receive the formatted data associated with a plurality of medical procedures for generation of one or more automated healthcare applications from data repository system 108. In some non-limiting embodiments, the formatted data may include formatted data associated with a plurality of medical procedures for generation of one or more machine learning models of an automated healthcare application.

In some non-limiting embodiments, data repository system 108 may receive initial data (e.g., raw data) associated with one or more data records via one or more data pipelines (e.g., extract, transform, load (etl) pipelines). In some non-limiting embodiments, data repository system 108 may establish one or more data pipelines to allow for the automated retrieval of data associated with one or more data records from data sources 104.

In some non-limiting embodiments, the data associated with one or more data records may include data associated with a patient medical procedure, such as data associated with an electronic medical record (e.g., an electronic patient record) from an EMR system, data associated with an image generated by a medical imaging system, data associated with a biometric measurement of a patient, data associated with a fluid injection procedure performed by a fluid injection system, data associated with a record from a pathology information system, and/or a laboratory information system. In some non-limiting embodiments, data associated with a patient medical procedure may include data associated with a fluid injection procedure, which may include data associated with a contrast fluid provided during a fluid injection procedure, a gauge of a catheter used during a fluid injection procedure, a fluid injection protocol for a fluid injection procedure, a configuration of a fluid injection system, and/or the like. Additionally or alternatively, data associated with a patient medical procedure may include data associated with a medical imaging procedure, which may include data associated with an image (e.g., a radiology image), data associated with radiation dosage, and/or the like. Additionally or alternatively, the data may include data (e.g., metadata) associated with identification of a patient, data associated with identification of a patient medical procedure (e.g., data regarding a medical procedure performed on a patient), data associated with identification of a device that is involved in a patient medical procedure (e.g., data associated with identification of a fluid injection system, data associated with identification of a device of a patient, etc.), and/or the like.

In some non-limiting embodiments, data repository system 108 may receive the initial data associated with one or more data records based on a device associated with data source 104 performing a medical procedure. For example, data repository system 108 may receive the data after the device associated with data source 104 performs a procedure. In some non-limiting embodiments, data repository system 108 may receive the data from a device associated with a hospital (e.g., a server operated by a hospital, such as a server that is on-site at a hospital or a server that is located remotely from a hospital, a hospital information system, etc.). In some non-limiting embodiments, data repository system 108 may store the data (e.g., with an application, in a memory device, etc.) based on receiving the data. In some non-limiting embodiments, data repository system 108 may receive the data (e.g., from a hospital information system) according to a communications protocol for communicating the data via a communication network. For example, data repository system 108 may receive the healthcare data according to a DICOM communications protocol, according to a Health Level Seven (HL7) standard communications protocol, and/or the like.

In some non-limiting embodiments, data repository system 108 may perform one or more data processing procedures based on receiving the data associated with one or more data records from data sources 104 to provide the formatted data associated with a plurality of medical procedures for generation of one or more automated healthcare applications (e.g., one or more machine learning models of an automated healthcare application). In some non-limiting embodiments, the data processing procedures may include one or more data curation procedures. In some non-limiting embodiments, the data curation procedures may include a de-identification procedure (e.g., a procedure to de-identify data associated with a data record, such as for removal of personal health information (PHI)), a quality check procedure, a data bias check procedure, a data conversion procedure, a de-duplication procedure, a data enhancement procedure (e.g., a procedure that applies preprocessing algorithms to raw data, a procedure that creates additional data, such as synthetic data, such as image rotation, a data balancing and/or data enrichment procedure, etc.), a data combination procedure, a data annotation procedure (e.g., a procedure by which data is labeled, such as drawing a bounding box around a site in an image), a data versioning procedure, and/or the like. Additionally or alternatively, data repository system 108 may perform one or more data transformation procedures based on receiving the data associated with one or more data records from data sources 104 to provide formatted data associated with a plurality of medical procedures for generation of an automated healthcare application. In some non-limiting embodiments, the one or more data transformation procedures may include a feature extraction procedure, a de-noising procedure, a redaction procedure (e.g., a text redaction procedure, where text is removed from a data record, an image redaction procedure, where at least a portion of an image is removed from a data record), and/or the like. In some non-limiting embodiments, the formatted data associated with generation of an automated healthcare application may include training data, validation data, and/or testing data for one or more machine learning models.

In some non-limiting embodiments, data repository system 108 may segment the data associated with one or more data records and/or the formatted data associated with generation of an automated healthcare application into a plurality of datasets for generation of automated healthcare applications. For example, data repository system 108 may segment the data associated with one or more data records into datasets based on performing one or more data processing procedures on the data. In some non-limiting embodiments, each dataset of the plurality of datasets may be associated with a particular medical procedure of the plurality of medical procedures.

In some non-limiting embodiments, AI medical device platform 102 and/or data repository system 108 may receive a request for access to a particular dataset of a plurality of datasets from a user associated with user device 106, determine whether the request for access complies with one or more criteria associated with access to the particular dataset, and provide access for user device 106 to the particular dataset based on determining that the request for access complies with one or more criteria associated with access to the particular dataset. In some non-limiting embodiments, AI medical device platform 102 and/or data repository system 108 may determine that the request for access complies with the criteria based on user identification information (e.g., a username, a password, an identification number, a device identifier associated with user device 106, such as an internet protocol (IP) address, media access control (MAC) address, etc.). For example, AI medical device platform 102 and/or data repository system 108 may determine whether the user identification information, provided as part of the access request, matches the one or more criteria associated with access to the particular dataset. AI medical device platform 102 and/or data repository system 108 may determine that the user identification information matches additional user identification information stored in a data structure. In some non-limiting embodiments, if AI medical device platform 102 and/or data repository system 108 determines that the request for access complies with the one or more criteria, then the user may be provided with access to the particular dataset. If AI medical device platform 102 and/or data repository system 108 determines that the request for access does not comply with the one or more criteria, then the user may not be provided with access to the particular dataset. In some non-limiting embodiments, the one or more criteria associated with access to the particular dataset may include whether the user has access to a subscription associated with the particular dataset. In some non-limiting embodiments, when providing access to the particular dataset, AI medical device platform 102 may perform an application programming interface (API) call associated with the particular dataset to data repository system 108 to provide access to the particular dataset.

As shown in FIG. 3, at step 304, process 300 includes receiving data associated with one or more regulatory guidance and approval processes. In some non-limiting embodiments, AI medical device platform 102 may receive the data associated with one or more regulatory guidance and approval processes from data repository system 108. In some non-limiting embodiments, the data associated with one or more regulatory guidance and approval processes may include standards (e.g., regulatory standards and/or non-regulatory standards) promulgated by a government entity, such as the United States Food and Drug Administration (FDA) and the Medical Device Coordination Group (MDCG) of the European Union, standards promulgated by non-government entities, such as the International Organization for Standardization (ISO) and the International Electrotechnical Commission (IEC). In some non-limiting embodiments, AI medical device platform 102 may receive the data associated with one or more regulatory guidance and approval processes from data repository system 108.

As shown in FIG. 3, at step 306, process 300 includes generating one or more automated healthcare applications configured to provide an output associated with a medical procedure. In some non-limiting embodiments, AI medical device platform 102 may generate the one or more automated healthcare applications based on the formatted data associated with a plurality of medical procedures for generation of one or more automated healthcare applications and/or the data associated with one or more regulatory guidance and approval processes. In some non-limiting embodiments, AI medical device platform 102 may generate one or more machine learning models of the one or more automated healthcare applications. In some non-limiting embodiments, the one or more automated healthcare applications (e.g., one or more machine learning models of the one or more automated healthcare applications) may be configured to provide an output associated with an aspect of (e.g., a result of) a medical procedure. In some non-limiting embodiments, the one or more automated healthcare applications may include a computer assisted detection application, and/or a computer assisted diagnosis application.

In some non-limiting embodiments, AI medical device platform 102 may generate one or more machine learning models. In some non-limiting embodiments, the machine learning model may include a machine learning model designed to receive, as an input, data, and provide, as an output, a prediction of a result (e.g., a prediction of a classification of an image, a prediction of the presence of a disease state, a prediction a treatment procedure, etc.) associated with a medical procedure. For example, the machine learning model may be designed to receive data (e.g., data associated with a patient procedure) and provide an output that includes the predicted classification of an automated healthcare data analysis application of a plurality of automated healthcare data analysis applications. The predicted classification of an automated healthcare data analysis application may include a classification of an automated healthcare data analysis application that may be able to provide further analysis of the healthcare data. In some non-limiting embodiments, AI medical device platform 102 may store the machine learning model (e.g., for later use).

In some non-limiting embodiments, as described herein, AI medical device platform 102 may process data (e.g., data associated with the plurality of medical procedures) to obtain training data (e.g., a training dataset) for the machine learning model. For example, AI medical device platform 102 may process the data to change the data into a format that may be analyzed (e.g., by AI medical device platform 102) to generate the machine learning model. The data that is changed (e.g., the data that results from the change) may be referred to as training data. In some non-limiting embodiments, AI medical device platform 102 may process the data to obtain the training data based on receiving the data. Additionally or alternatively, AI medical device platform 102 may process the data to obtain the training data based on AI medical device platform 102 receiving an indication, from a user (e.g., a user associated with user device 106) of AI medical device platform 102, that AI medical device platform 102 is to process the data, such as when AI medical device platform 102 receives an indication to generate a machine learning model (e.g., for a time interval corresponding to the data, for a location corresponding to the data, for a device corresponding to the data, etc.).

In some non-limiting embodiments, AI medical device platform 102 may process data by determining a prediction variable based on the data. A prediction variable may include a metric, associated with one or more medical procedures of a plurality of medical procedures, which may be derived based on the data. The prediction variable may be analyzed to generate a machine learning model. For example, the prediction variable may include a variable associated with a patient procedure (e.g., a time of a medical procedure, a protocol used during a medical procedure, an amount of a substance used during a medical procedure, a result of a medical procedure, etc.), a variable associated with an aspect of image (e.g., a radiological image), a variable associated with a patient demographic, a variable associated with a condition of a patient, and/or the like.

In some non-limiting embodiments, AI medical device platform 102 may analyze the training data to generate the machine learning model. For example, AI medical device platform 102 may use machine learning techniques to analyze the training data to generate the machine learning model. In some non-limiting embodiments, generating the machine learning model may be referred to as training the machine learning model. The machine learning techniques may include, for example, supervised and/or unsupervised techniques, such as decision trees, random forests, logistic regressions, linear regression, gradient boosting, support-vector machines, extra-trees (e.g., an extension of random forests), Bayesian statistics, learning automata, Hidden Markov Modeling, linear classifiers, quadratic classifiers, association rule learning, computer vision, and/or the like. In some non-limiting embodiments, the machine learning model may include a model that is specific to a particular characteristic, for example, a model that is specific to a particular location (e.g., a specific location of a hospital), a particular patient demographic, a particular system used during a patient procedure, and/or the like. Additionally or alternatively, the machine learning model may be specific to a particular entity (e.g., a healthcare provider, such as a hospital, a clinical facility, a group of doctors, etc.) that provides healthcare services. In some non-limiting embodiments, AI medical device platform 102 may generate one or more machine learning models for one or more entities, a particular group of entities, and/or one or more users of one or more entities.

Additionally or alternatively, when analyzing the training data, AI medical device platform 102 may identify one or more variables (e.g., one or more independent variables) as predictor variables (e.g., features) that may be used to make a prediction when analyzing the training data. In some non-limiting embodiments, values of the predictor variables may be inputs to the machine learning model. For example, AI medical device platform 102 may identify a subset (e.g., a proper subset) of the variables as the predictor variables that may be used to accurately predict a result associated with a medical procedure. In some non-limiting embodiments, the predictor variables may include one or more of the prediction variables, as discussed above, that have a significant impact (e.g., an impact satisfying a threshold) on a prediction as determined by AI medical device platform 102.

In some non-limiting embodiments, AI medical device platform 102 may validate the machine learning model. For example, AI medical device platform 102 may validate the machine learning model after AI medical device platform 102 generates the machine learning model. In some non-limiting embodiments, AI medical device platform 102 may validate the machine learning model based on a portion of the training data to be used for validation. For example, AI medical device platform 102 may partition the training data into a first portion and a second portion, where the first portion may be used to generate the machine learning model, as described above. In this example, the second portion of the training data (e.g., the validation data) may be used to validate the machine learning model.

In some non-limiting embodiments, AI medical device platform 102 may validate the machine learning model by providing validation data as input to the machine learning model, and determining, based on an output of the machine learning model, whether the machine learning model correctly, or incorrectly, predicted a result associated with a medical procedure. In some non-limiting embodiments, AI medical device platform 102 may validate the machine learning model based on a validation threshold. For example, AI medical device platform 102 may be configured to validate the machine learning model when the result associated with a medical procedure (as identified by the validation data) is correctly predicted by the machine learning model (e.g., when the machine learning model correctly predicts 50% of the results, 70% of the results, a threshold quantity of the results, and/or the like).

In some non-limiting embodiments, if AI medical device platform 102 does not validate the machine learning model (e.g., when a percentage of correctly predicted results associated with a medical procedure does not satisfy the validation threshold), then AI medical device platform 102 may generate one or more additional machine learning models.

In some non-limiting embodiments, once the machine learning model has been validated, AI medical device platform 102 may further train the machine learning model and/or generate new machine learning models based on receiving new training data. The new training data may include additional data associated with a plurality of medical procedures. In some non-limiting embodiments, the new training data may include new healthcare data associated with a plurality of patient procedures (e.g., data associated with a plurality of patient procedures that were performed following a specified time). AI medical device platform 102 may use the machine learning model to predict a result associated with a medical procedure and compare an output of machine learning models to the new training data that includes data associated with a plurality of medical procedures. In such an example, AI medical device platform 102 may update one or more machine learning models based on the new training data.

In some non-limiting embodiments, AI medical device platform 102 may store the machine learning model. For example, AI medical device platform 102 may store the machine learning model in a data structure (e.g., a database, a linked list, a tree, and/or the like). The data structure may be located within AI medical device platform 102 or external (e.g., remote from) AI medical device platform 102, such as within data repository system 108.

In some non-limiting embodiments, when generating an automated healthcare application that is configured to predict one or more aspects associated with a medical procedure, AI medical device platform 102 may train one or more machine learning models of the automated healthcare application based on the data associated with the plurality of medical procedures to provide one or more trained machine learning models and AI medical device platform 102 may validate an output of the one or more trained machine learning models based on the data associated with the regulatory guidance and approval process. In some non-limiting embodiments, AI medical device platform 102 may validate the output by comparing the output to a standard included in the data associated with the regulatory guidance and approval process. If the output satisfies the standard, AI medical device platform 102 may validate the one or more trained machine learning models. If the output does not satisfy the standard, AI medical device platform 102 may forego validating the one or more trained machine learning models.

In some non-limiting embodiments, AI medical device platform 102 may train the one or more machine learning models based on a first dataset of the plurality of datasets to provide one or more trained machine learning models, and AI medical device platform 102 may assign a unique identifier to the first dataset based on training the one or more machine learning models and assign the unique identifier to the one or more trained machine learning models. In some non-limiting embodiments, when validating the output of the one or more trained machine learning models, AI medical device platform 102 may validate the first dataset and the one or more trained machine learning models based on the unique identifier assigned to the first dataset and the one or more trained machine learning models. For example, AI medical device platform 102 may validate the first dataset and the one or more trained machine learning models by comparing the unique identifier assigned to the first dataset and the unique identifier assigned to the one or more trained machine learning models. If the unique identifier assigned to the first dataset and the unique identifier assigned to the one or more trained machine learning models correspond, AI medical device platform 102 may validate the one or more trained machine learning models. If the unique identifier assigned to the first dataset and the unique identifier assigned to the one or more trained machine learning models do not correspond, AI medical device platform 102 may forego validating the one or more trained machine learning models.

As shown in FIG. 3, at step 308, process 300 includes deploying the one or more automated healthcare applications to a production environment. For example, AI medical device platform 102 may deploy the one or more automated healthcare applications to the production environment based on generating the one or more automated healthcare applications.

In some non-limiting embodiments, AI medical device platform 102 may deploy the one or more automated healthcare applications to a production environment of a third-party platform that is not associated with AI medical device platform 102 (e.g., a third-party platform that is operated by an entity that is not associated with an entity that operates AI medical device platform 102), a production environment of the AI medical device platform, a platform that includes a standardized container, or any combination thereof.

In some non-limiting embodiments, prior to deploying an automated healthcare application, AI medical device platform 102 may perform a clinical evaluation of the automated healthcare application based on the data associated with the one or more regulatory guidance and approval processes. For example, AI medical device platform 102 may perform a performance assessment of the automated healthcare application using clinical test data to determine whether the automated healthcare application satisfies a regulatory standard. In some non-limiting embodiments, the performance assessment may include a retrospective reader study (e.g., a study involving clinical test data) and/or a prospective reader study (e.g., a study involving assessment based on data acquired during the study). In some non-limiting embodiments, AI medical device platform 102 may generate one or more reports based on a result of the performance assessment.

In some non-limiting embodiments, AI medical device platform 102 may receive a request for access to a particular automated healthcare application (e.g., one or more machine learning models of an automated healthcare application) of a plurality of automated healthcare applications from a user associated with user device 106, determine whether the request for access complies with one or more criteria associated with access to the particular automated healthcare application, and provide access for user device 106 to the particular automated healthcare application based on determining that the request for access complies with one or more criteria associated with access to the particular automated healthcare application. In some non-limiting embodiments, AI medical device platform 102 may determine that the request for access complies with the criteria based on user identification information (e.g., a username, a password, an identification number, a device identifier associated with user device 106, such as an internet protocol (IP) address, media access control (MAC) address, etc.). For example, AI medical device platform 102 may determine whether the user identification information, provided as part of the access request, matches the one or more criteria associated with access to the particular automated healthcare application. AI medical device platform 102 may determine that the user identification information matches additional user identification information stored in a data structure. In some non-limiting embodiments, if AI medical device platform 102 determines that the request for access complies with the one or more criteria, then the user may be granted access to the particular automated healthcare application. If AI medical device platform 102 determines that the request for access does not comply with the one or more criteria, then the user may not be granted access to the particular automated healthcare application. In some non-limiting embodiments, the one or more criteria associated with access to the particular automated healthcare application may include whether the user has access to a subscription associated with the particular dataset.

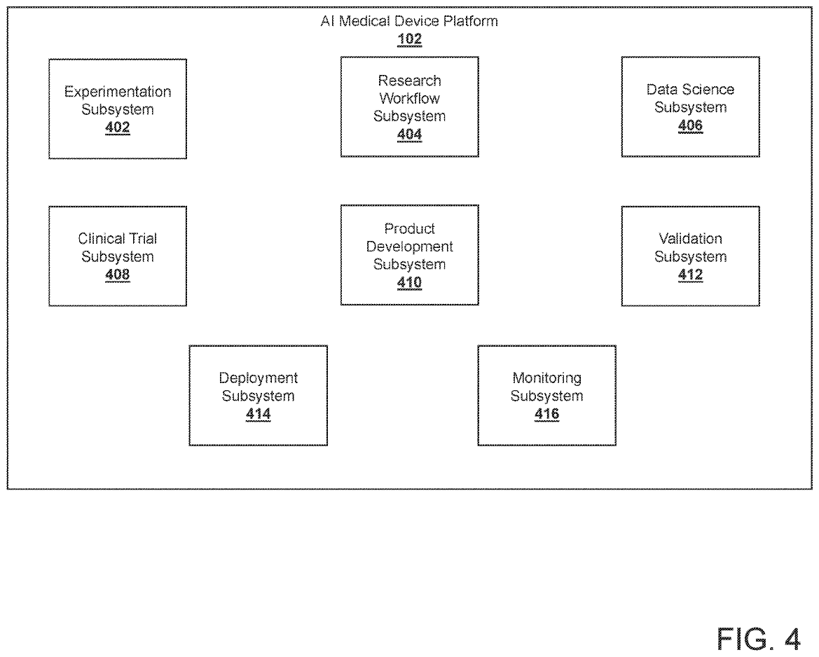

Referring now to FIG. 4, FIG. 4 is a diagram of a non-limiting embodiment of AI medical device platform 102. In some non-limiting embodiments, AI medical device platform 102 may include a plurality of subsystems (e.g., hardware components, software components, such as modules, applications, extensions, units, etc., that are configured to be executed on one or more devices of AI medical device platform 102 or any combination thereof). In some non-limiting embodiments, the plurality of subsystems of AI medical device platform 102 may interconnect with each other via wired connections, wireless connections, or a combination of wired and wireless connections.

As shown in FIG. 4, the plurality of subsystems may include experimentation subsystem 402, research workflow subsystem 404, data science subsystem 406, clinical trials subsystem 408, product development subsystem 410, validation subsystem 412, deployment subsystem 414, and monitoring subsystem 416. In some non-limiting embodiments, one or more subsystems of the plurality of subsystems of AI medical device platform 102 may be hosted on a network device (e.g., a network edge device) that is closest to a facility that interacts with AI medical device platform 102. In this way, results from the one or more subsystems of AI medical device platform 102 may be received in less time as compared to a situation where the one or more subsystems are hosted on a different network device. In some non-limiting embodiments, AI medical device platform 102 may include more than or less than the plurality of subsystems shown in FIG. 4. In some non-limiting embodiments, AI medical device platform 102 may include one or more subsystems that are configured to be operated based on a manually provided configuration (e.g., a configuration provided by a user, such as a doctor, technician, etc.).

In some non-limiting embodiments, experimentation subsystem 402 may be configured to receive and/or provide specification data associated with one or more aspects of a medical procedure to allow for development of (e.g., a concept for) one or more machine learning models (e.g., one or more automated healthcare applications that include one or more machine learning models) associated with the medical procedure and/or to allow for generation of a proof of concept for one or more machine learning models associated with the medical procedure. In some non-limiting embodiments, experimentation subsystem 402 may provide recommendations for one or more machine learning models associated with a medical procedure. For example, experimentation subsystem 402 may provide recommendations for concepts for one or more machine learning models associated with a medical procedure based on inputs received from a user that include the specification data.

In some non-limiting embodiments, experimentation subsystem 402 may receive data associated with a concept for an automated healthcare application. For example, experimentation subsystem 402 may receive data associated with a concept for a machine learning model that provides an output that is a prediction of a treatment outcome. In some non-limiting embodiments, experimentation subsystem 402 may generate data associated with a plurality of machine learning models (e.g., a plurality of types of machine learning models, a plurality of machine learning model architectures, a plurality of parameters associated with machine learning models, etc.) based on the data associated with the concept for the automated healthcare application. For example, experimentation subsystem 402 may provide a list of a plurality of machine learning models. In some non-limiting embodiments, experimentation subsystem 402 may receive a selection (e.g., a user selection) of one or more machine learning models and experimentation subsystem 402 may generate data associated with a proof of concept for the automated healthcare application.

In some non-limiting embodiments, research workflow subsystem 404 may be configured to provide automation support and/or data enrichment procedures. In some non-limiting embodiments, research workflow subsystem 404 may provide data enrichment procedures such as identification of anatomical regions of a body in an image, image quality modifications, such as adjustments for noise in an image, blurring in an image, and/or text detection in an image.

In some non-limiting embodiments, data science subsystem 406 may be configured to provide standard software tools and/or libraries, procedures for automation of continuous integration and continuous delivery/deployment (CI/CD), procedures for abstraction from infrastructure, medical image processing functions, model training functions, procedures for machine learning paradigms, including federated learning, procedures for machine learning model cataloging and/or machine learning model sharing, and/or procedures for collaboration for development of machine learning models.

In some non-limiting embodiments, clinical trial subsystem 408 may be configured to receive, store, and/or provide data associated with a clinical trial of an automated healthcare application. In some examples, clinical trial subsystem 408 may receive, store and/or provide data associated with evaluation of an automated healthcare application (e.g., product evaluation, product evaluation design, etc.), data associated with clinical site setup, data associated with an evaluation system setup, and/or data associated with inputs and/or outputs provided to and/or received from an automated healthcare application (e.g., image data de-identification, image data transfer, image data quality check, image data curation and/or preparation). In some non-limiting embodiments, clinical trial subsystem 408 may be accessible separately from other subsystems of AI medical device platform 102. For example, clinical trial subsystem 408 may be separated from other subsystems by a data firewall.

In some non-limiting embodiments, product development subsystem 410 may be configured to provide data associated with software development tools, an integrated validated toolchain, data associated with user feedback, data associated with issue tracking, data associated with regulatory documentation, data associated with technical documentation, a code repository, an artifact repository, tools for data tracking, tools for model tracking, tools for experiment tracking, and/or data associated with pipelining of data.

In some non-limiting embodiments, validation subsystem 412 may be configured to provide the ability to determine whether an automated healthcare application satisfies regulatory requirements associated with an output of the automated healthcare application (e.g., an output of a machine learning model that includes a prediction regarding one or more aspects associated with a healthcare procedure, such as predicted treatment, a predicted diagnosis, a predicted image for an imaging procedure, etc.).

In some non-limiting embodiments, validation subsystem 412 may be configured to provide the ability to determine whether an automated healthcare application satisfies regulatory requirements associated with an output of the automated healthcare application (e.g., an output of a machine learning model that includes a prediction regarding one or more aspects associated with a medical procedure, such as predicted treatment, a predicted diagnosis, a predicted image for an imaging procedure, etc.).

In some non-limiting embodiments, deployment subsystem 414 may be configured to provide the ability to deploy an automated healthcare application in a production environment. In some examples, deployment subsystem 414 may provide for containerization of the automated healthcare application, container management of containers for automated healthcare applications, continuous deployment procedures for automated healthcare applications, data integration with other services (e.g., including other AI based services, such as Bayer Calantic™ Digital Solutions), and/or integration with developer portals for other services (e.g., including other AI based services, such as Bayer Calantic™ Digital Solutions).

In some non-limiting embodiments, monitoring subsystem 416 may be configured to provide the ability to monitor an automated healthcare application that is deployed in a production environment. For example, monitoring subsystem 416 may be configured to receive, store, and/or provide analysis of outputs of an automated healthcare application that is deployed in a production environment. In some non-limiting embodiments, monitoring subsystem 416 may be configured to monitor an automated healthcare application for issues associated with data drift, issues associated with model drift, automated healthcare application usage information, and/or for information associated with life cycle management of an automated healthcare application. In some non-limiting embodiments, monitoring subsystem 416 may be configured to monitor an automated healthcare application based on a predetermined time interval (e.g., an hourly time interval, a daily time interval, a weekly time interval, a monthly time interval, etc.). In some non-limiting embodiments, monitoring subsystem 416 may be configured to determine an accuracy of an automated healthcare application and compare the accuracy to a threshold value of accuracy (e.g., a threshold value of accuracy based on a government regulation, a threshold value based on a key performance indicator (KPI) of accuracy). If monitoring subsystem 416 determines that the accuracy of the automated healthcare application satisfies the threshold value of accuracy, monitoring subsystem 416 may allow the automated healthcare application to remain online (e.g., remain online in a production environment). If monitoring subsystem 416 determines that the accuracy of the automated healthcare application does not satisfy the threshold value of accuracy, monitoring subsystem 416 may take the automated healthcare application offline (e.g., remove the automated healthcare application from a production environment).

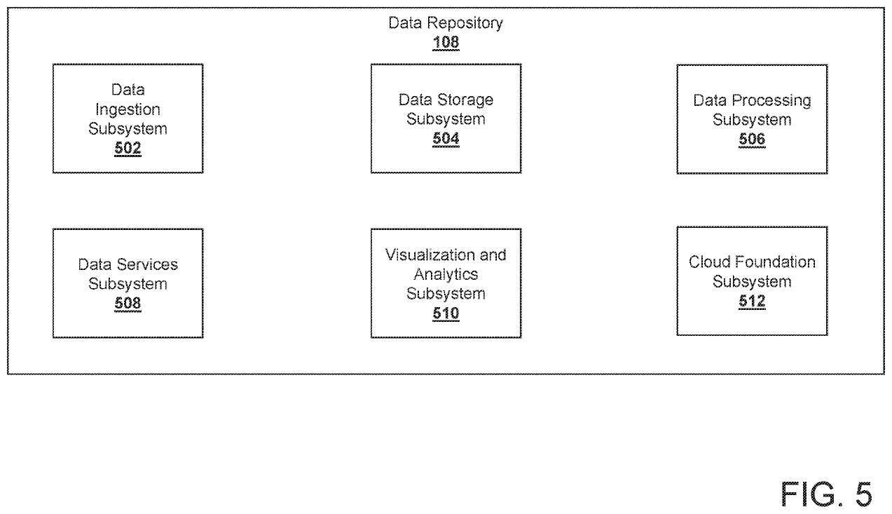

Referring now to FIG. 5, FIG. 5 is a diagram of a non-limiting embodiment of data repository system 108. In some non-limiting embodiments, data repository system 108 may include a plurality of subsystems (e.g., hardware components, software components, such as modules, applications, extensions, units, etc., that are configured to be executed on one or more devices of data repository system 108 or any combination thereof). In some non-limiting embodiments, the plurality of subsystems of data repository system 108 may interconnect with each other via wired connections, wireless connections, or a combination of wired and wireless connections.

As shown in FIG. 5, the plurality of subsystems may include data ingestion subsystem 502, data storage subsystem 504, data processing subsystem 506, data services subsystem 508, visualization and analytics subsystem 510, and cloud foundation subsystem 512. In some non-limiting embodiments, one or more subsystems of the plurality of subsystems of data repository system 108 may be hosted on a network device (e.g., a network edge device) that is closest to a facility that interacts with data repository system 108. In this way, results from the one or more subsystems of data repository system 108 may be received in less time as compared to a situation where the one or more subsystems are hosted on a different network device. In some non-limiting embodiments, data repository system 108 may include more than or less than the plurality of subsystems shown in FIG. 5. In some non-limiting embodiments, data repository system 108 may include one or more subsystems that are configured to be operated based on a manually provided configuration (e.g., a configuration provided by a user, such as a doctor, technician, etc.).

In some non-limiting embodiments, data ingestion subsystem 502 may be configured to receive initial data (e.g., raw data) associated with one or more data records from a plurality of data sources (e.g., data sources 104). In some non-limiting embodiments, data ingestion subsystem 502 may create temporary virtual machine instances to ingest data from on-premises servers (e.g., via secure file transfer protocol (SFTP) and/or file transfer protocol (FTP)) and/or cloud storage platforms. Jobs performed by data ingestion subsystem 502 may be recurring according to predetermined time intervals.

In some non-limiting embodiments, data storage subsystem 504 may be configured to function as a primary source of data for data repository system 108. In some non-limiting embodiments, data storage subsystem 504 may distinguish between data from different data sources and/or data in different formats and store the data in a memory device.

In some non-limiting embodiments, data processing subsystem 506 may be configured to process initial data associated with one or more data records received from a plurality of data sources received via one or more data pipelines. In some non-limiting embodiments, data processing subsystem 506 may transform the initial data based on extract, transform, load (etl) pipelines to provide transformed data to be used for generation of one or more automated healthcare applications (e.g., one or more machine learning models of one or more automated healthcare applications).

In some non-limiting embodiments, data services subsystem 508 may be configured to provide for manual and/or automated access to data stored in data repository system 108. In some non-limiting embodiments, data services subsystem 508 may be configured to provide services related to viewing data in the form of images, including an image viewer, services for generating ground truth information for data, services that define protocols for collecting data, services related to generating data, including generative AI algorithms. In some non-limiting embodiments, the services provided by data services subsystem 508 may be based on clinical procedures.

In some non-limiting embodiments, visualization and analytics subsystem 510 may be configured to provide access to data visualization tools and/or database analytics tools to be used for generation of one or more automated healthcare applications (e.g., one or more machine learning models of one or more automated healthcare applications). In some non-limiting embodiments, visualization and analytics subsystem 510 may include services to perform online analytical processing (OLAP) on data and to search and retrieve particular data of interest to a user.

In some non-limiting embodiments, cloud foundation subsystem 512 may be configured to control operations related to data repository system 108. For example, cloud foundation subsystem 512 may provide one or more rules associated with control operations (e.g., authentication, authorization, network and security, data encryption, monitoring and logging aspects, etc.) for data repository system 108.

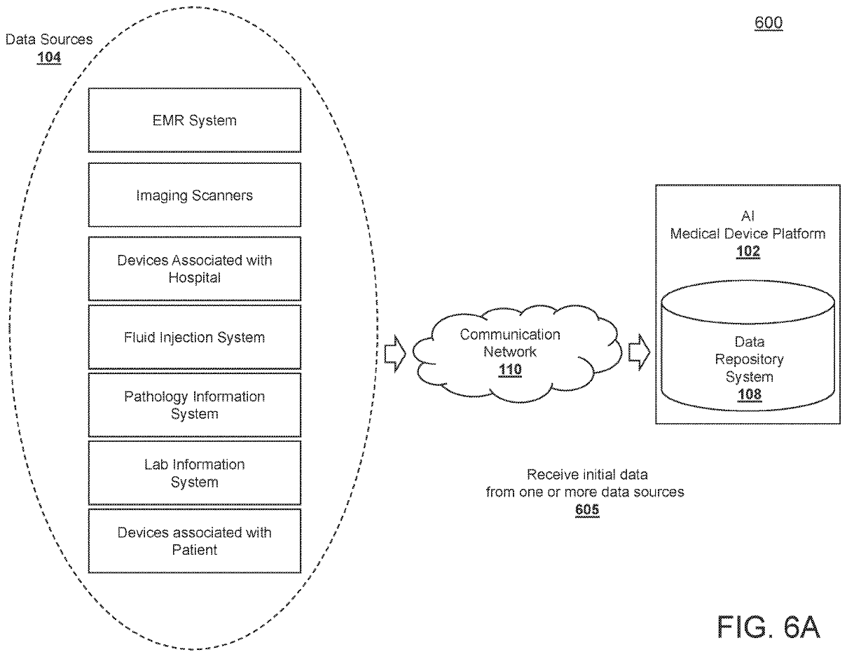

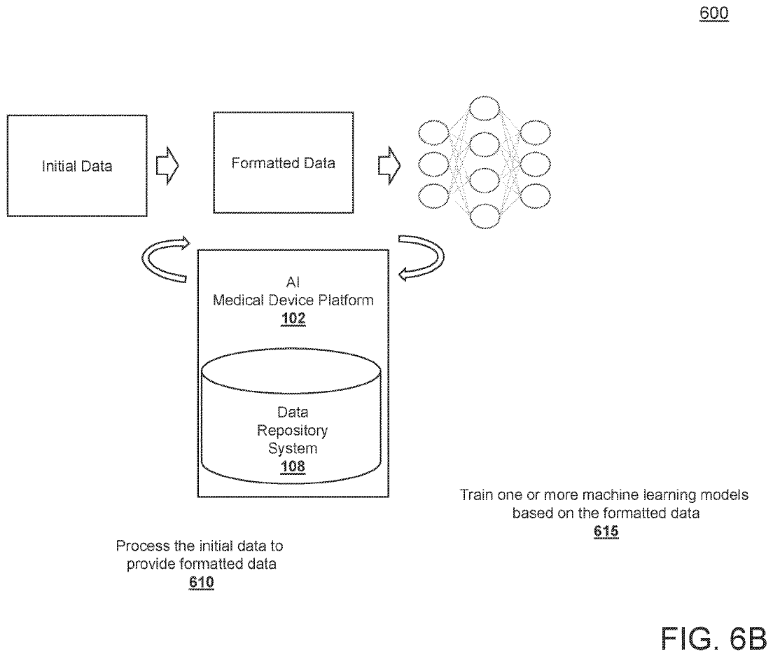

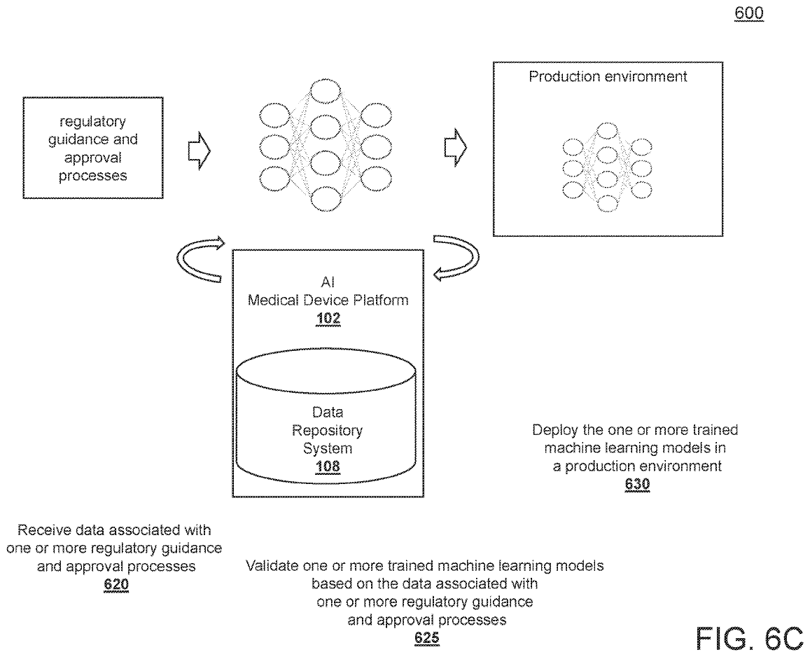

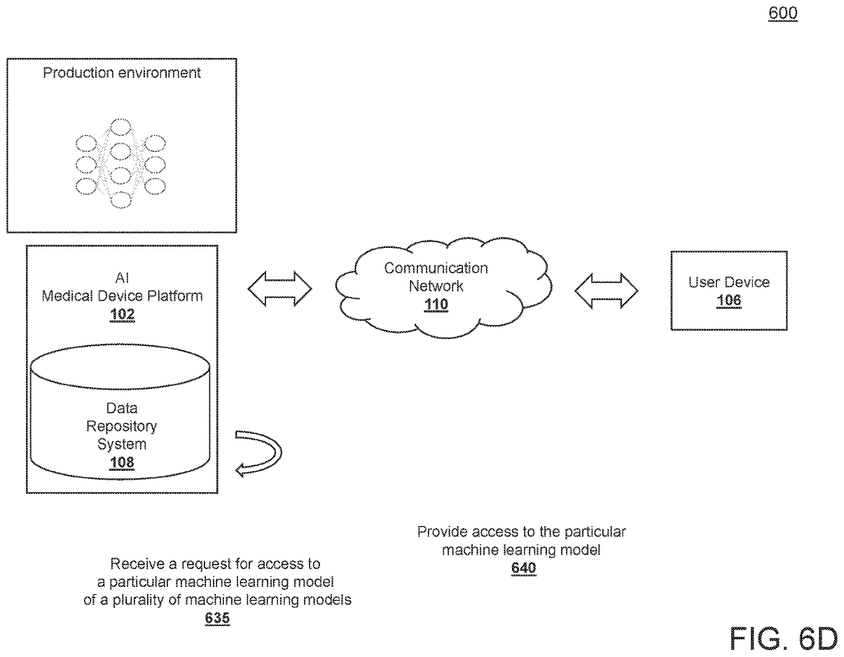

Referring now to FIGS. 6A-6D, FIGS. 6A-6D are diagrams of a non-limiting embodiment of implementation 600 of a process (e.g., process 300) for generation of AI based automated healthcare applications using an AI medical device platform.

As shown by reference number 605 in FIG. 6A, AI medical device platform 102 may receive initial data from data sources 104. In some non-limiting embodiments, the initial data may include initial data (e.g., raw data) associated with one or more data records of one or more medical procedures.

As shown by reference number 610 in FIG. 6B, AI medical device platform 102 may process the initial data to provide formatted data. In some non-limiting embodiments, AI medical device platform 102 may process the initial data by performing one or more data curation procedures on the initial data to provide the formatted data. In some non-limiting embodiments, the formatted data may include formatted data associated with a plurality of medical procedures for generation of one or more machine learning models. As further shown by reference number 615 in FIG. 6B, AI medical device platform 102 may train one or more machine learning models based on the formatted data.

As shown by reference number 620 in FIG. 6C, AI medical device platform 102 may receive data associated with one or more regulatory guidance and approval processes. In some non-limiting embodiments, AI medical device platform 102 may receive the data associated with one or more regulatory guidance and approval processes from data repository system 108. In some non-limiting embodiments, the data associated with one or more regulatory guidance and approval processes may include standards (e.g., regulatory standards and/or non-regulatory standards) promulgated by a government entity and/or standards promulgated by non-government entities.

As further shown by reference number 625 in FIG. 6C, AI medical device platform 102 may validate one or more trained machine learning models based on the data associated with one or more regulatory guidance and approval processes. As further shown by reference number 630 in FIG. 6C, AI medical device platform 102 may deploy the one or more trained machine learning models in a production environment.

As shown by reference number 635 in FIG. 6D, AI medical device platform 102 may receive a request for access to a particular machine learning model of a plurality of machine learning models. As shown by reference number 640 in FIG. 6D, AI medical device platform 102 may provide access to the particular machine learning model.

Although the above systems, methods, and computer program products have been described in detail for the purpose of illustration based on what is currently considered to be the most practical and preferred embodiments, it is to be understood that such detail is solely for that purpose and that the present disclosure is not limited to the described embodiments but, on the contrary, is intended to cover modifications and equivalent arrangements that are within the spirit and scope of the appended claims. For example, it is to be understood that the present disclosure contemplates that, to the extent possible, at least one feature of any embodiment or aspect can be combined with at least one feature of any other embodiment or aspect.

1. A system for generation of artificial intelligence (AI) based automated healthcare applications, comprising:
a data repository system, wherein the data repository system stores at least one of the following: formatted data associated with a plurality of medical procedures for generation of one or more machine learning models, data associated with a regulatory guidance and approval process, or any combination thereof;
an AI medical device platform that comprises at least one processor in communication with the data repository system, the at least one processor configured to:
receive the formatted data associated with a plurality of medical procedures for generation of one or more machine learning models;
receive the data associated with one or more regulatory guidance and approval processes;
generate one or more machine learning models configured to predict one or more aspects associated with a medical procedure based on the formatted data associated with the plurality of medical procedures and the data associated with the regulatory guidance and approval process;
deploy the one or more machine learning models to a production environment based on generating the one or more machine learning models.

2. The system of claim 1, wherein the data repository system comprises:
a data ingestion subsystem to receive data from a plurality of data sources;
a data storage subsystem to store data for the data repository system;
a data processing subsystem to transform the data of the plurality of data sources based on extract, transform, load (etl) pipelines to provide transformed data to be used for generation of the one or more machine learning models;
a data analytics subsystem to provide access to data visualization tools and database analytics tools to be used for generation of the one or more machine learning models; and
a data cloud foundation subsystem to provide one or more rules associated with control operations for the data repository system.

3. The system of claim 1, wherein the data repository system is configured to:
receive initial data associated with one or more data records from a plurality of data sources, wherein the plurality of data sources comprises at least one of the following:
an electronic medical record (EMR) system;
a medical imaging system;
a fluid injection system;
a pathology information system;
a laboratory information system; or
any combination thereof; and
perform one or more data curation procedures on the initial data associated with one or more data records to provide the formatted data associated with a plurality of medical procedures for generation of one or more machine learning models.

4. The system of claim 1, wherein, when generating one or more machine learning models configured to predict one or more aspects associated with a medical procedure, the at least one processor is configured to:
train the one or more machine learning models based on the formatted data associated with a plurality of medical procedures for generation of one or more machine learning models to provide one or more trained machine learning models; and
validate an output of the one or more trained machine learning models based on the data associated with the regulatory guidance and approval process.

5. The system of claim 1, wherein the at least one processor is further configured to:
receive a request for access to a particular machine learning model of a plurality of machine learning models;
determine whether the request for access complies with one or more criteria associated with access to the particular machine learning model; and
provide access to the particular machine learning model based on determining that the request for access complies with one or more criteria associated with access to the particular machine learning model.

6. The system of claim 1, wherein the formatted data associated with a plurality of medical procedures for generation of one or more machine learning models comprises a plurality of datasets, wherein each dataset of the plurality of datasets is associated with a particular medical procedure of the plurality of medical procedures, wherein the at least one processor is further configured to:
receive a request for access to a particular dataset of a plurality of datasets;
determine whether the request for access complies with one or more criteria associated with access to the particular dataset; and
provide access to the particular dataset based on determining that the request for access complies with one or more criteria associated with access to the particular dataset.

7. The system of claim 6, wherein, when providing access to the particular dataset, the at least one processor is configured to:
perform an application programming interface (API) call associated with the particular dataset to the data repository system to provide access to the particular dataset.

8. The system of claim 1, wherein the formatted data associated with a plurality of medical procedures for generation of one or more machine learning models comprises a plurality of datasets, wherein each dataset of the plurality of datasets is associated with a particular medical procedure of the plurality of medical procedures, wherein the at least one processor is further configured to:
train the one or more machine learning models based on a first dataset of the plurality of datasets to provide one or more trained machine learning models;
assign a unique identifier to the first dataset based on training the one or more machine learning models;
assign the unique identifier to the one or more trained machine learning models; and
validate an output of the one or more trained machine learning models based on the data associated with the regulatory guidance and approval process, wherein when validating the output of the one or more trained machine learning models, the at least one processor is configured to:
validate the first dataset and the one or more trained machine learning models based on the unique identifier assigned to the first dataset and the one or more trained machine learning models.

9. The system of claim 1, wherein, when deploying the one or more machine learning models to the production environment, the at least one processor is configured to:
deploy the one or more machine learning models to at least one of the following:
a production environment of a third-party platform that is not associated with the AI medical device platform;
a production environment of the AI medical device platform;
a platform that includes a standardized container; or
any combination thereof.

10. A method for generation of artificial intelligence (AI) based automated healthcare applications, comprising:
receiving, with at least one processor of an AI medical device platform, formatted data associated with a plurality of medical procedures for generation of one or more machine learning models from a data repository system;
receiving, with the at least one processor, data associated with one or more regulatory guidance and approval processes;
generating, with the at least one processor, one or more machine learning models configured to predict one or more aspects associated with a medical procedure based on the formatted data associated with a plurality of medical procedures for generation of one or more machine learning models and the data associated with the regulatory guidance and approval process; and
deploying, with the at least one processor, the one or more machine learning models to a production environment based on generating the one or more machine learning models.

11-18. (canceled)

19. A computer program product for generation of artificial intelligence (AI) based automated healthcare applications comprising at least one non-transitory computer-readable medium including program instructions that, when executed by at least one processor, cause the at least one processor to:
receive formatted data associated with a plurality of medical procedures for generation of one or more machine learning models from a data repository system;
receive data associated with one or more regulatory guidance and approval processes from the data repository system;
generate one or more machine learning models configured to predict one or more aspects associated with a medical procedure based on the data associated with the plurality of medical procedures and the data associated with the regulatory guidance and approval process; and
deploy the one or more machine learning models to a production environment based on generating the one or more machine learning models.

20. The computer program product of claim 19, wherein the program instructions further cause the at least one processor to:
receive initial data associated with one or more data records from a plurality of data sources, wherein the plurality of data sources comprises at least one of the following:
an electronic medical record (EMR) system;
a medical imaging system;
a fluid injection system;
a pathology information system;
a laboratory information system;
any combination thereof.

21. The computer program product of claim 20, wherein the program instructions further cause the at least one processor to:
perform one or more data curation procedures on the initial data associated with one or more data records to provide the formatted data associated with a plurality of medical procedures for generation of one or more machine learning models.

22. The computer program product of claim 19, wherein the program instructions that cause the at least one processor to generate one or more machine learning models configured to predict one or more aspects associated with a medical procedure cause the at least one processor to:
train the one or more machine learning models based on the formatted data associated with a plurality of medical procedures for generation of one or more machine learning models to provide one or more trained machine learning models; and
validate an output of the one or more trained machine learning models based on the data associated with the regulatory guidance and approval process.

23. The computer program product of claim 19, wherein the program instructions further cause the at least one processor to:
receive a request for access to a particular machine learning model of a plurality of machine learning models;
determine whether the request for access complies with one or more criteria associated with access to the particular machine learning model; and
provide access to the particular machine learning model based on determining that the request for access complies with one or more criteria associated with access to the particular machine learning model.

24. The computer program product of claim 19, wherein the formatted data associated with a plurality of medical procedures for generation of one or more machine learning models comprises a plurality of datasets, wherein each dataset of the plurality of datasets is associated with a particular medical procedure of the plurality of medical procedures, wherein the program instructions further cause the at least one processor to:
receive a request for access to a particular dataset of a plurality of datasets;
determine whether the request for access complies with one or more criteria associated with access to the particular dataset; and
provide access to the particular dataset based on determining that the request for access complies with one or more criteria associated with access to the particular dataset.

25. The computer program product of claim 24, wherein the program instructions that cause the at least one processor to provide access to the particular dataset cause the at least one processor to:
perform an application programming interface (API) call associated with the particular dataset to the data repository system to provide access to the particular dataset.

26. The computer program product of claim 19, wherein the formatted data associated with a plurality of medical procedures for generation of one or more machine learning models comprises a plurality of datasets, wherein each dataset of the plurality of datasets is associated with a particular medical procedure of the plurality of medical procedures, wherein the program instructions further cause the at least one processor to:
train the one or more machine learning models based on a first dataset of the plurality of datasets to provide one or more trained machine learning models;
assign a unique identifier to the first dataset based on training the one or more machine learning models;
assign the unique identifier to the one or more trained machine learning models; and
validate an output of the one or more trained machine learning models based on the data associated with the regulatory guidance and approval process, wherein the program instructions that cause the at least one processor to validate the output of the one or more trained machine learning models cause the at least one processor to:
validate the first dataset and the one or more trained machine learning models based on the unique identifier assigned to the first dataset and the one or more trained machine learning models.

27. The computer program product of claim 19, wherein the program instructions that cause the at least one processor to deploy the one or more machine learning models to the production environment cause the at least one processor to:
deploy the one or more machine learning models to at least one of the following:
a production environment of a third-party platform that is not associated with the AI medical device platform;
a production environment of the AI medical device platform;
a platform that includes a standardized container; or
any combination thereof.
